(19) United States
(12) Patent Application Publication
(10) Pub. No.: US 2026/0253664 A1
(43) Pub. Date: Aug. 27, 2026

(54) ARTIFICIAL INTELLIGENCE ASSISTED COMPUTATIONAL FRAGMENT-BASED DRUG DESIGN

(71) Applicant: Drexel University, Philadelphia, PA (US)

(72) Inventors: Hai-feng Ji, Cherry Hill, NJ (US); Gail Rosen, Philadelphia, PA (US); Bahrad Sokhansanj, Los Angeles, CA (US); Wei Chuen Chong, Philadelphia, PA (US); Jerica Wilson, Frederick, MD (US); Rohan Chandraghatgi, Philadelphia, PA (US)

(21) Appl. No.: 19/160,068

(22) PCT Filed: Feb. 28, 2024
(86) PCT No.: PCT/US24/17640

§ 371 Date: Aug. 27, 2025

Related U.S. Application Data
(60) Provisional application No. 63/448,899, filed on Feb. 28, 2023.

(51) Int. Cl. G16B 15/30; G16H 70/40

(52) U.S. Cl. CPC G16B 15/30; G16H 70/40

(57) ABSTRACT

Provided herein are methods for improving drug discovery using artificial intelligence to identify compounds that are expected to have potent binding affinities at therapeutic targets of interest.

### CROSS-REFERENCE TO RELATED APPLICATION

This application claims the benefit of priority to U.S. Provisional Patent Application Ser. No. 63/448,899, entitled “ARTIFICIAL INTELLIGENCE BASED COMPUTATIONAL FRAGMENT-BASED DRUG DESIGN,” filed Feb. 28, 2023, the disclosure of which is incorporated herein by reference in its entirety.

### BACKGROUND

Fewer than 1 out of 10 drug candidates garner FDA approval, an amount disproportionate to the volume of resources employed in the drug discovery process. Drug discovery is an extensive and costly process bearing low yield. Even the fraction of drugs that do make it to market will have spent 10-15 years of time in development with a $1-2 billion cost.

Recently, “fragment-based drug discovery” (or “design”) (FBDD) has emerged as a potentially promising approach. Unlike other drug design strategies (i.e., structure-based drug discovery, SBDD, or ligand-based drug discovery, LBDD), which usually involve designing or testing full-sized drug molecules, works by identifying smaller chemical fragments that bind effectively to target biomolecules.

However, despite the potential advantages of FBDD, a significant challenge remains: the scale of the chemical space to be explored. Given the enormous diversity of possible chemical fragments and the ways they can be combined, the number of potential drug candidates is effectively infinite. This massive combinatorial problem can become a stumbling block, slowing down the drug discovery process and making it difficult to identify promising candidates.

The present disclosure provides a solution to this unmet need.

### BRIEF SUMMARY OF THE INVENTION

In one aspect, a computer-implemented method of drug design is provided. The method incudes the steps of:
(a) accessing a computer model of an atomic structure of a target protein and a plurality of ligands independently docked and positioned in a binding region in the target protein, wherein each ligand in the plurality of ligands has an associated binding affinity with the target protein;
(b) fragmenting each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally joining two or more ligand fragments to form a drug, wherein a chemical structure of the drug is chemically stable and the two or more ligand fragments have fragment binding scores in the top 10% of a rank-ordered sorting of ligand binding scores of the plurality of ligand fragments.

In one aspect, a system configured to implement the methods described herein is provided. The system includes:
a computer system comprising one or more processors;
the computer system being configured and adapted to implement deep reinforcement learning, the computer system being further configured and adapted to be provided with objectives for a therapeutically effective drug, wherein the one or more processors are configured to execute a set of instructions that:
   (a) provide a computer model of an atomic structure of a target protein and a plurality of ligands independently docked in a binding region in the target protein, wherein each ligand in the plurality of ligands has an associated binding affinity with the target protein;
   (b) fragment each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
   (c) computationally join two or more ligand fragments to form a drug, wherein a chemical structure of the drug is chemically stable and the two or more ligand fragments have fragment binding scores in the top 10% of a rank-ordered sorting of ligand binding scores of the plurality of ligand fragments.

In one aspect, a computer-implemented method of drug design is provided. The method includes:
(a) accessing a computer model of a target protein and computer models of a plurality of ligand structures;
(b) obtaining a predicted binding affinity between each of the ligands and the target protein;
(c) providing protein-ligand bond profiling data based on computational predictions on how each of the plurality of ligand structures binds with the target protein;
(d) fragmenting the plurality of ligand structures into computer models of ligand fragments; and
(e) providing scores for each of the ligand fragments using the predicted binding affinity and the protein-ligand bond profiling data.

In various aspects, a computer-implemented method of drug design is provided. The method includes:
(a) accessing a computer model of an atomic structure of a target protein and a plurality of ligands independently docked in a binding region in the target protein, wherein each ligand in the plurality of ligands has an associated binding affinity with the target protein;
(b) fragmenting each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally joining two or more ligand fragments to form a drug, wherein a chemical structure of the drug is chemically stable and the two or more ligand fragments have fragment binding scores above a certain threshold of a rank-ordered sorting of ligand binding scores of the plurality of ligand fragments.

### BRIEF DESCRIPTION OF THE DRAWINGS

For a fuller understanding of the nature and desired objects of the present invention, reference is made to the following detailed description taken in conjunction with the accompanying drawing figures.

FIG. 1 illustrates fragments binding to particular regions, in accordance with exemplary embodiments of the present disclosure.

FIG. 2 is a schematic showing processing of prescreened ligands, according to various embodiments. The prescreening begins with the output from prescreening a library of ligands with Autodock VINA, selecting the minimum binding affinity structure. PLIP (Protein-Ligand Interaction Profiler) predicts the binding residues based on the docked ligand-protein complex using a rules-based heuristic algorithm. The ligands are computationally fragmented using the BRICS algorithm. The output from this workflow is a table fragments, identified by their SMILES representation, associated with the binding pocket residues identified by PLIP, as well as the median binding affinity of the parent ligands and the frequency in which the combination is found in the prescreened library. Notably, a fragment will appear multiple times if it is found to bind to different residues (i.e., binding pocket subdomains) in the context of different prescreened ligands.

FIG. 3 scheme used for generating fragments, in accordance with exemplary embodiments of the present disclosure.

FIG. 4 illustrates the most frequently occurring fragments for each protein of a study conducted in accordance with exemplary embodiments of the present disclosure.

FIG. 5 illustrates structures of top 3 ligands from HTVS method (top) and top 3 ligands from FDSL (bottom), in accordance with exemplary embodiments of the present disclosure.

FIGS. 6A-6F illustrate various 3D and 2D representations of interactions of screened ligands vs FLDD ligands, in accordance with exemplary embodiments of the present disclosure.

FIGS. 7A-7C illustrate the top ten most frequently occurring fragments from highest 10% of binding ligands for each protein TIPE2 (FIG. 7A), RelA (FIG. 7B), and S-protein (FIG. 7C), respectively, in accordance with various embodiments of the present disclosure.

FIG. 8 illustrates a flow diagram of a method of drug design, in accordance with exemplary embodiments of the present disclosure.

FIG. 9 illustrates a flow diagram of a method of drug design, in accordance with exemplary embodiments of the present disclosure.

FIG. 10 illustrates an example of a final generated ligand and the fragments fed into the BRICS algorithm to generate it, according to various embodiments.

FIG. 11 shows molecular structures of potential candidate ligands generated by the FDSL and two-stage optimization pipeline, according to various embodiments. Candidate TIPE2 ligands: (i), (ii), and (iii). Candidate RelA ligands: (iv), (v), and (vi). Candidate Spike RBD ligands: (vii), (viii), and (ix).

FIG. 12 shows a schematic of ligand optimization, according to various embodiments. In the first phase of ligand optimization, a genetic algorithm is used to create ligands from the fragments produced by the prescreening and fragmentation pipeline shown in FIG. 1. Fragments are represented like genes and assigned a weighted rank to determine selection probability. Initially, fragments are randomly chosen and evaluated using Autodock VINA (see “Autodock Vina” block) and optionally analyzed for drug likeness via QED scores. Subsequent ligand generations are crafted using mutation (“Mutation” block), crossover, and elitism strategies, abiding by specific molecular weight and fragment use rules. The ligands are further cleaned to ensure all fragments are utilized in the resultant ligand (“Clean Ligands” block) and constructed into new generations using the BRICS.BUILD module in RDKit (“BRICS.Build Generates Ligands” block).

FIG. 13 shows a schematic of the iterative fragment addition stage, according to various embodiments. The iterative fragment addition stage can begin with any kind of starting ligand but in our method begins with candidate ligands synthesized through the previous genetic algorithm-based ligand synthesis phase (see FIG. 2) and fragments obtained from the initial ligand prescreening and fragmentation phase (see FIG. 1). The optimization objective of this phase is the binding affinity score predicted by AutoDock VINA, alone or in a sum with the Quantitative Effectiveness of Druglikeness (QED) score, which evaluates beneficial molecular properties beneficial for drug design. The methodology begins with premade starter ligands and an amino acid-associated fragment dataset. Through successive iterations, each ligand is evaluated and possibly merged with a protein PDB file for further assessment by the Protein-Ligand Interaction Profiler (PLIP). Fragments are strategically added to target regions of the ligand, ensuring optimal binding affinity and maintaining molecular weights under 700 g/mol to ensure viable drug targets. This process cyclically refines ligand structures, using tools like RDKit for optimization and 3D structuring, continuing to a prescribed iteration limit or until an optimizable ligand is generated.

FIG. 14 shows histograms comparing fragment pool generation methodologies, according to various embodiments, for the TIPE2, RelA, and Spike RBD targets. The top three graphs include comprehensive results of each iterative run. The “Worst Pool” trial used the worst 1000 fragments by VINA score from the source fragment dataset. The “Large Pool” included all fragments. The “Unprioritized” and “Prioritized” trials used the same subset of fragments generated with priority of VINA score and top 1000 fragments associated with a given amino acid. Unprioritized trials use randomly assigned fragments in the pool to bind to the ligands, while Prioritized trials use sub-pools for each amino acid. For a given target amino acid, the Prioritized suggested a fragment known to have interacted with that amino acid in the past based off the PLIP screening.

FIG. 15 shows a comparison of AutoGrow4 and iterative generated ligands. The proposed method's histogram data was selected from Prioritized run described in FIG. 14 and accompanying text.

FIG. 16 shows the plotted mean VINA score of each iteration in the Deep Frag runs, according to various embodiments. The unbiased standard error of the mean is calculated for each iteration and displayed as error bars. Scores tend to increase per iteration of DeepFrag, indicating worsened binding affinities. The best VINA scores for each protein target are −12.17, −11.47, and −9.895 kcal/mol for TIPE2, RelA, and Spike RBD respectively. The graph is plotted such that the y-axis values decrease from bottom to top to show stronger binding affinities as higher values. Also, the y-axis range differs for each target to illustrate the similarity in trend between the targets differently for each target.

FIG. 17 shows histograms comparing multi-objective and VINA prioritizations over final VINA and QED scores using iterative approach. Lower VINA scores indicate improved binding affinity and higher QED scores indicate better drug-likeness. Red lines indicate 50th and 95th percentile scores, which are selected to segment regions of each dataset. Both iterative runs are run on the same set of starting ligands from the genetic algorithm, which is run with multi-objective prioritization. The starting ligands are selected based on best multi-objective score.

FIG. 18 illustrates the strongest ligand candidates, with respect to predicted binding affinity, the histograms shown here plot the 95th percentile ligands by VINA score with QED score for each of the protein targets. Horizontal line indicates 97.5th percentile VINA score. Vertical line indicates the 50th percentile QED score of the 95th percentile ligands by VINA score.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

As used herein, the term “fragment” can refer to a small molecule that typically consists of a low number of atoms (in various embodiments less than 10, 15, or 20 atoms, not including hydrogen) and represents a simplified version of a larger molecule. The term “fragment” can also refer to a contiguous portion of a ligand. In some contexts, a fragment may be below a size threshold, or a fragment may be defined as a part of a ligand that can include some of its chemical activity. As an example, “fragments” can be used in fragment-based drug discovery (FBDD) as a starting point to identify small molecules that can bind to a target protein or enzyme, and which can then be elaborated into larger and more potent drug-like compounds.

As used herein, the term “ligand” can refer to a molecule that binds to a specific target (e.g., molecule), typically a protein or enzyme. In many examples, a “ligand” (when bound to a target) can modulate the activity of a target (e.g., activation or inhibition of enzymatic activity, modulation of protein-protein interactions, or stabilization of protein conformation).

As used herein, “docking” refers to computational simulation of a candidate ligand binding to a receptor.

As used herein, the term “drug-like” refers to any of the following, or a combination of any of the following:
i) a compound that contains no more than 5 hydrogen bond donors, no more than 10 hydrogen bond acceptors, a molecular mass less than 600 g/mol, and a calculated octanol-water partition coefficient (C log P) that is 5 or less;
ii) a compound that has a C log P between about-0.4 and about 5.6, a molecular refractivity from about 40 to about 130, a molecular mass between 180 and 600, and total number from about 20 to about 80; or
iii) ten or less rotatable bonds and a polar surface area of 140 Å² or less;
iv) W log P less than 6 and polar surface area less than about 135 Å²; or
v) a molecular mass between 200 and 600 g/mol, X log P between about −2 and about 5, polar surface area less than about 150 Å², 7 or fewer rings, more than 4 carbons, more than 1 heteroatoms, less than 15 rotatable bonds, less than 10 hydrogen bond acceptors, and less than 5 hydrogen bond donors.

As used herein, a “rotatable bond” is any single bond, not in a ring, bound to a non-terminal heavy (i.e., non-hydrogen) atom and not including amide C—N bonds.

As used herein, the term “polar surface area” or “total polar surface area” (TPSA) refers to the area of a molecule's surface belonging to polar atoms, as described in, for example, Ertl, P. et al. J. Med. Chem. 2000, 43, 20, 3714-3717.

As used herein, the term “chemically stable” means a compound that does not further react, isomerize, or decompose under one or more of the following conditions: a) temperatures of 10 to 40° C.; b) a relative humidity of 30 to 100%; c) when formulated in a pharmaceutical composition containing one or more pharmaceutically acceptable excipients as described herein. In various embodiments, a chemically stable compound is stable for a period of one week to one year, or more. In various embodiments, a chemically stable compound is stable in the solid state and/or in a liquid formulation.

In certain embodiments, an artificial neural network with the ability to dock fragments of organic molecules into a biological target has been developed.

Fragment based drug discovery (FBDD) is a drug discovery technique used to identify and develop new lead compounds for a biological target. Screening is conducted using low molecular weight (<300 amu) organic compounds, considered fragments. These fragments are bound to the biological target and the binding affinity is determined followed by structural determinations using X-ray crystallography. Fragments can then be linked together to yield a higher binding lead compound, or a high binding fragment can be built upon.

Only 15% of drugs that originate from pharmaceutical Research and Discovery are approved, with almost half of designed drugs failing in late stages. A call for new methods is paramount to mitigate such low success. Artificial Intelligence (AI) can be used in the drug discovery process, expanding the prospects for discovering and designing new therapeutic options. Incorporating AI with a fragment-based discovery approach can lead to heightened success in identifying potential inhibitors.

Computational drug discovery has the potential to leverage the power of in silico screening by using machine learning and optimization techniques to predict the bioactivity of potential compounds and, thereby, accelerate drug discovery. Recently, “fragment-based drug discovery” (or “design”) (FBDD) has emerged as a potentially promising approach. Unlike other drug design strategies (i.e., structure-based drug discovery, SBDD, or ligand-based drug discovery, LBDD), which usually involve designing or testing full-sized drug molecules, works by identifying smaller chemical fragments that bind effectively to target biomolecules. These small fragments serve as building blocks, which can be grown, linked, or merged to create new drug molecules. FBDD offers a flexible and efficient way to explore the vast potential space of drug-like molecules.

However, despite the potential advantages of FBDD, a significant challenge remains: the scale of the chemical space to be explored. Given the enormous diversity of possible chemical fragments and the ways they can be combined, the number of potential drug candidates is effectively infinite. This massive combinatorial problem can become a stumbling block, slowing down the drug discovery process and making it difficult to identify promising candidates. In various embodiments, the methods herein provide computational generation of potential drug molecules. Computational de novo drug design involves the use of techniques such as genetic algorithms, reinforcement learn-ing, including deep reinforcement learning, generative deep learning models, or other deep learning methods, e.g., graph transformers, models that blend deep learning and evolutionary algorithms, and string-based trans-formers (i.e., operating on a SMILES string representation of molecules).

The algorithms “computationally synthesize” new drug molecules, either by starting from scratch and adding atoms to form a new molecule or modifying or adding atoms on an existing chemical structure (“scaffold”). The result is the creation of new molecules by a) simulating chemical modifications that optimize for the single objective of improving binding efficiency to a target or b) multi-objective optimization including drug-likeness objectives, e.g., solubility and other drug-likeness factors. In computational FBDD, various in silico computational techniques are utilized to construct fragment libraries for Fragment-Based Drug Discovery (FBDD).

The conventional approach to computational FBDD involves either computationally fragmentizing a com-pound (ligand) library or self-generating fragments using computational techniques, followed by computationally docking target fragments to a protein binding pocket and computationally “growing” or synthesizing a candidate ligand by modifying the fragment within that pocket. Methods like FastGrow emphasize identifying fragment growth points rather than the specifics of fragment expansion, often comparing to other structural docking tools. In the realm of docking, ultra-large scale docking techniques, such as those by Lyu et al., identify potential molecules based on docking scores, with a breadth possibly surpassing human intuition.

The advent of “deep evolutionary learning” for FBDD introduced the employment of a latent space grounded in SMILES, as seen in methods like FragVAE, which incorporates evolutionary operators and data augmentation in the process. Subsequent techniques, such as Podda et al.'s encoder-decoder generative model, also employ the SMILES structure to produce fragments. More advanced strategies integrate graph-based and evolutionary operators on a molecule's latent representation, focusing on multi-objective optimization.

Leveraging either heuristic (evolutionary) approaches or learning techniques, these methods aim to identify superior optima. However, given the expansive nature of the chemical space, any strategy will fail to be a universally optimal solution to all possible problems and chemical configurations. In addition to the limits on heuristic optimization methods, deep learning methods are limited because training typically probes only a minute subspace of the chemical structure landscape. To reduce the combinatorial search space and achieve more targeted drug designs, a pipeline for computational FBDD described herein called Fragment Databases from Screened Ligands Drug Discovery (FDSL-DD) is utilized. The FDSL pipeline involves an initial in silico screening step of a large ligand database against a protein target, utilizing computational docking software, e.g., Autodock VINA.

The ligands are then computationally fragmented, and the fragments are assigned information (i.e., fragment attributes) based on the predicted binding affinity (docking score) and amino acids that are predicted to interact with atoms in the computational fragment. The information output from the FDSL pipeline can then be used to resynthesize the fragments in new combinations and generate synthetic ligands which could form the basis for potential candidate lead compounds. The FDSL approach thereby contrasts with conventional computational FDBB, which, even when based on predetermined ligand libraries, do not retain information about protein-ligand binding based on initial virtual library screening. For that reason, FDSL constrains the optimization space to identify the best possible trajectories for fragment growth and virtual compound synthesis, and thus can more readily identify promising lead designs.

Described herein in various embodiments is a computational drug design methodology that employs two stages of optimization based on applying the fragments as well as the associated fragment attributes derived from ligand prescreening. The two optimization stages, according to various embodiments, include:
1) evolutionary optimization uses principles of natural selection and genetic variation in a computational method for strategically guiding the assembly of synthetic fragments to generate larger compounds; and
2) iterative optimization refines the resulting compounds by adding small fragments to improve bioactivity.

At both stages, the fragment information obtained from the FDSL pipeline narrows down the vast chemical space by imposing constraints that limit the search to areas with higher potential for success. Using fragment attributes will both reduce the combinatorial size of the search space and focus the search for ligands on potentially more favorable parts of the space. The resulting computational drug design process not only becomes more efficient, but also more likely to yield compounds with high binding affinities and desirable drug-like properties.

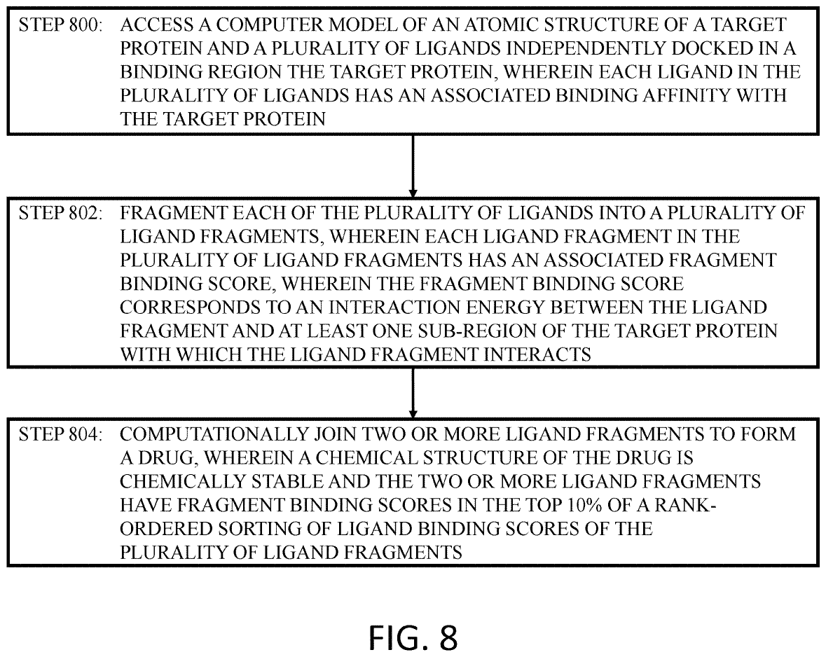

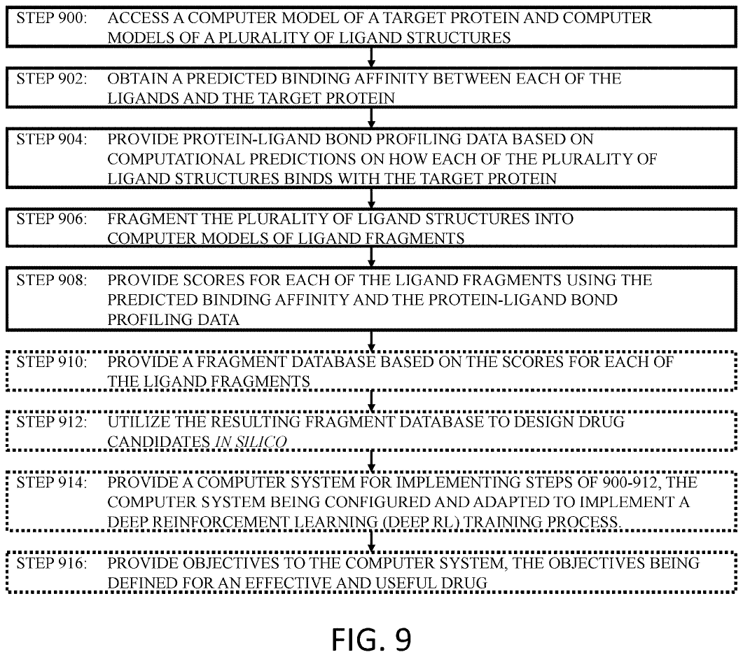

FIGS. 8 and 9 are flow diagrams in accordance with various embodiments of the methods described herein. As is understood by those skilled in the art, certain steps included in the flow diagrams may be omitted; certain additional steps may be added; and the order of the steps may be altered from the order illustrated.

In various embodiments, referring to FIG. 8, a method of drug designed is illustrated. In Step 800, a computer model of an atomic structure of a target protein and a plurality of ligands independently docked in a binding region the target protein are accessed, wherein each ligand in the plurality of ligands has an associated binding affinity with the target protein. In Step 802, each of the plurality of ligands is fragmented into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts. In Step 804, two or more ligand fragments are computationally joined to form a drug, wherein a chemical structure of the drug is chemically stable and the two or more ligand fragments have fragment binding scores in the top 10% of a rank-ordered sorting of ligand binding scores of the plurality of ligand fragments.

In certain applications, the drug is physically (i.e., not merely in a computerized form) synthesized and implemented in an experiment or used to treat a subject. Accordingly, a certain number of the top compounds, as determined by the methods of the present disclosure, can be implemented. For example, the top three candidate compounds can be implemented to treat a target. In another example, the top 10% of candidate compounds can be implemented to treat a target.

In various embodiments, referring now to FIG. 9, a method of drug designed is illustrated. In Step 900, a computer model of a target protein and computer models of a plurality of ligand structures are accessed. In Step 902, a predicted binding affinity between each of the ligands and the target protein are obtained. In Step 904, protein-ligand bond profiling data, based on computational predictions on how each of the plurality of ligand structures binds with the target protein, are provided. In Step 906, the plurality of ligand structures are fragmented into computer models of ligand fragments. In Step 908, scores for each of the ligand fragments are provided using the predicted binding affinity and the protein-ligand bond profiling data. In Step 910, a fragment database based on the scores for each of the ligand fragments is provided. In Step 912, the resulting fragment database is utilized to design drug candidates in silico. In Step 914, a computer system for implementing Steps of 900-912, is provided, the computer system being configured and adapted to implement a deep reinforcement learning (deep RL) training process. In Step 916, objectives are provided to the computer system, the objectives being defined for an effective and useful drug.

According to the present disclosure, a system to design and construct drugs (e.g., to treat a patient with a disease) is provided herein. In certain embodiments, the specific drugs are ligands, relatively small (e.g., generally <500 atomic molecular units) chemical compounds. Such small molecule drugs treat a disease by binding with a specific protein target to inhibit its function in patients. According to the present disclosure, an approach can be based on assembling fragments to formulate chemical structures for drug candidates that can bind well to drug targets while being conducive to delivery and transport in the human body. The entire contents of all patents, published patent applications, and other references cited herein are hereby expressly incorporated herein in their entireties by reference.

According to the present disclosure, a computer model of the atomic structure of a protein, that has been pre-determined to be a target to treat a particular disease, can be provided. Then computer models of viable ligand structures (e.g., a few hundred thousand) can be obtained. Then a computational docking software (e.g., Autodock) can be used to obtain the predicted binding affinity (e.g., strength of bonds) between each of the ligands and the protein targets. Software can be used to computationally predict where and how (i.e., what chemical bonds at what atoms) each ligand binds with the protein. After these screening and profiling steps, an algorithm that simulates the breaking of bonds within a ligand to synthesize computational fragments, such as the BRICS or RECAP rule-based fragmentation algorithms known to those skilled in the art, can be used to virtually “break up” the ligands into computer models of ligand fragments. The binding affinity and protein-ligand bond profiling data can be used from the screening step to provide scores for each of the fragments. The resulting fragment database can be utilized to design drug candidates in silico.

The present disclosure provides a computer system developed based on deep reinforcement learning (Deep RL). The system can be provided with objectives for an effective and useful drug. A main objective can be to achieve a high binding affinity with the protein target. Other important objectives can be to make the drug highly soluble and minimally hydrophobic, which can be necessary to provide a drug that can be practically delivered within the human body. Certain embodiments of the system can be directed to learn the best assemblies of fragments to formulate synthetic ligands (e.g., candidate drug designs) that meet defined objectives. The system can be engineered to “learn” how to optimize design by automatically reinforcing designs that better meet the objectives in the Deep RL training process.

The present disclosure provides a computer system that utilizes genetic or evolutionary algorithms both alone and in combination with Deep RL. The system can be provided with objectives for an effective and useful drug. A main objective can be to achieve a high binding affinity with the protein target. Other important objectives can be to make the drug highly soluble and minimally hydrophobic, which can be necessary to provide a drug that can be practically delivered within the human body. Genetic and evolutionary algorithms are methods for solving optimization problems that utilize algorithms based on the natural selection process underlying the evolution of organisms. In a genetic or evolutionary algorithm, potential ligands can be represented by assemblies of fragments to formulate synthetic ligands (e.g., candidate drug designs) which constitute individual solutions, which in turn are randomly modified (“mutated”) or recombined to produce superior solutions to achieve designed objectives. The system can be engineered to optimize design by selecting designs that better meet the objectives of the genetic or evolutionary optimization process.

Certain embodiments of such a system have shown the system can produce synthetic ligands with superior binding affinity to any of the original ligands that were started with in the screening step described herein. Such a system has demonstrated that the system can create the blueprints for synthetic ligands that can form the basis for treatments for different diseases for at least three different protein targets: PD-L1 (cancer), RelA (cancer and immune diseases), and the SARS-CoV-2 virus Spike glycoprotein (COVID-19).

As one example, tumor necrosis factor α-induced protein 8 like 2 or TIPE2 is a protein involved in leukocyte polarization. Controlling both the formation of the leading and trailing edge of a polarized leukocyte, TIPE2 initiates a cascade of events that lead to the sustainment of chronic inflammation, an environment known to allow tumor cells to thrive. Design of an inhibitor for TIPE2 would provide a therapeutic option for solid tumor cancers by preventing the framework necessary for tumor cell proliferation, migration and survival. Using certain methods and systems described herein, stronger inhibitors have been found for the proangiogenic TIPE2 protein (e.g., by utilizing a method of the present disclosure that combines both AI and FBDD).

Computer aided drug discovery can be used to expedite the drug discovery process. Structure based drug design (SBDD), which includes fragment-based drug design (FBDD), is a major drug discovery method employed in computer-aided drug discovery. Both SBDD and FBDD have limitations and new methods are paramount to mitigate low success rates. To effectively reduce the amount of time spent in pre-clinical phases, computer aided approaches can be incorporated into the major drug design strategies, with a more recent integration of artificial intelligence (AI), increasing the prospects for discovering and designing new therapeutic options. The present disclosure provides a Fragment Databases from Screened Ligands Drug Discovery (FDSL-DD) method that incorporates reinforcement learning into a fragment-based design approach to the drug development process.

Fragment-based drug design (FBDD) can utilize small molecules (molecular weight <300 g/mol), or fragments, to design a lead compound. Identified fragments can be grown, linked, or merged into a more potent lead molecule. In certain embodiments of the present disclosure, a linking method can be used for the FLDD as the approach is super-additive, with the binding energy of the linked molecule exceeding the sum binding energy of the fragments. The initial steps of such a FLDD deviate from the traditional in silico FBDD strategy, incorporating structure-based design screening techniques to combine the advantages of both approaches. Using drug-like ligands in the initial high throughput virtual screening allows for a greater search of chemical space, with less grid boxes, potentially identifying more interactions. Moreover, ligand screening can supply additional information that allows for more effective generation of candidate ligands. The number of fragment combinations and their orientations in the generation of ligands is combinatorically explosive. As a result, FBDD remains a challenge, since the space for identifying an effective drug candidate is still very large and finding candidates that are both feasible (e.g., drug-like) and high binding affinity to the target is a difficult task.

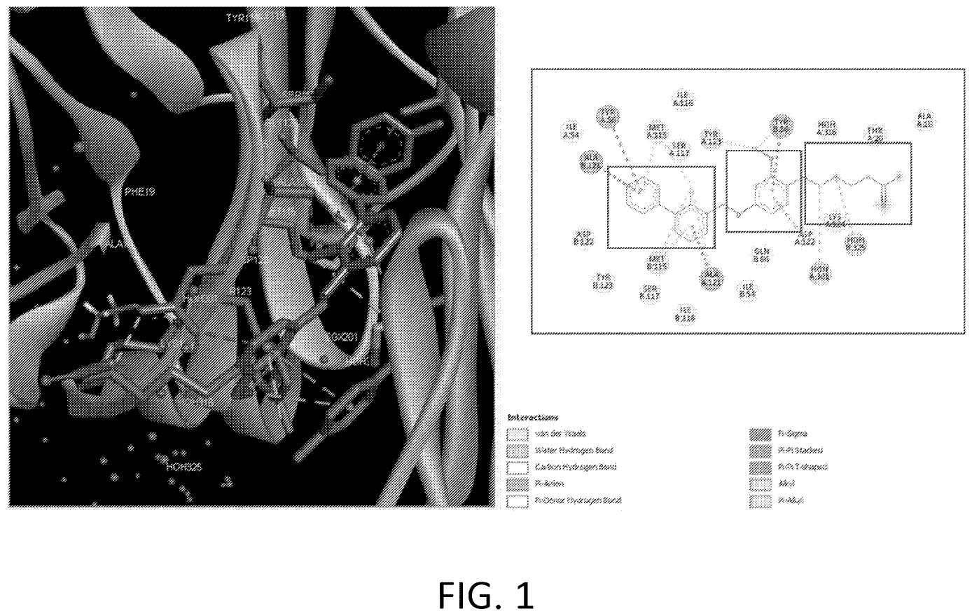

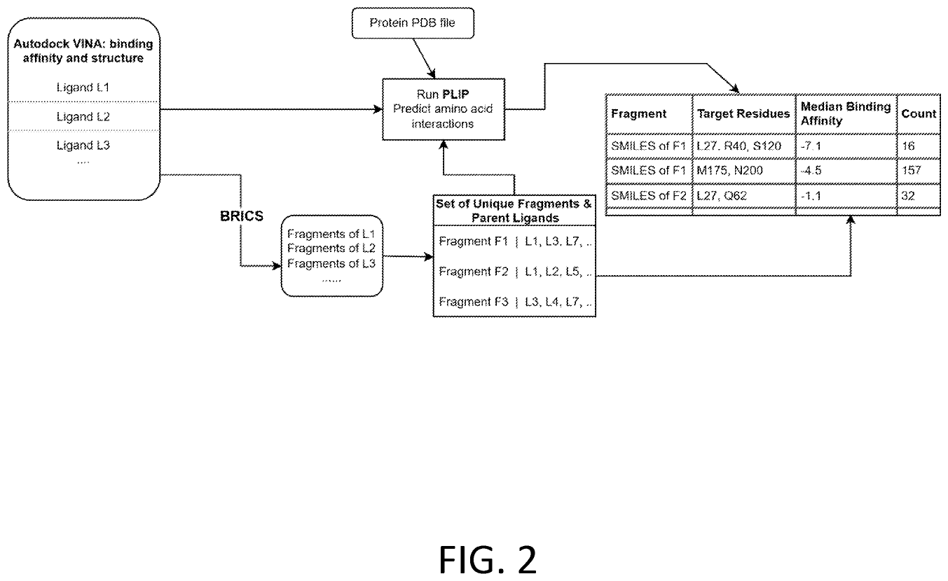

To the opposite of the traditional FBDD method, certain embodiments of the present disclosure are intended to adopt a new fragment-based method by creating a fragment database from a large, already docked, ligand screening library for a specific target, in which fragments are associated with information from the parent ligand (e.g., see FIG. 1). At a high level, many “drug like” ligands (e.g., preferably in the scale of millions) can be screened with computational docking software to obtain the predicted binding affinity between each of the ligands and the protein targets. The compounds with high binding scores can be then used to analyze where and how (i.e., what chemical bonds at what atoms) each ligand binds with the protein. After these screening and profiling steps, the ligands can be computationally fragmented (i.e., virtually broken up into fragments). A database can then be created which includes, for each fragment, summary statistics for the binding affinity of parent ligands and protein-ligand bond profiling data from the screening step. The fragments which appear more often than others will be identified as the top lead fragments (TLF) for the specific subpocket of the protein. The resulting fragment database can then be utilized to design drug candidates in silico by linking the top lead fragments (TLF) for the continuous subpockets to form a more potent inhibiting molecule (e.g., see FIG. 2).

Creating a fragment database from a large, already docked, ligand screening library, can be achieved using artificial intelligence and deep learning method(s) as described herein. Such methods can be used in the drug discovery process and expand the prospects for discovering and designing new therapeutic options. Incorporating AI with a fragment-based discovery approach can lead to heightened success in identifying potential inhibitors.

In various embodiments, a computer-implemented method of drug design is provided and includes the steps of:
(a) accessing a computer model of an atomic structure of a target protein and a plurality of ligands independently docked and positioned in a binding region in the target protein, wherein each ligand in the plurality of ligands has an associated binding affinity with the target protein;
(b) fragmenting each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally joining two or more ligand fragments to form a drug, wherein a chemical structure of the drug is chemically stable and the two or more ligand fragments have fragment binding scores in the top 10% of a rank-ordered sorting of ligand binding scores of the plurality of ligand fragments. In various embodiments, the two or more ligand fragments have fragment binding scores in the top 1, 2, 3, 4, 5, 6, 7, 8, 9, 10, 11, 12, 13, 14, or 15% of a rank-ordered sorting of ligand binding scores of the plurality of ligand fragments.

In various embodiments, the average molecular weight of the plurality of ligands is at least 350 g/mol. In various embodiments, the position of the plurality of ligands relative to the target protein is stored in a computer accessible database. In various embodiments, the method includes associating the plurality of ligand fragments with the ligand from which they originate.

In various embodiments, the position of the plurality of ligands fragments relative to the target protein is stored in a computer accessible database. The position can be, in various embodiments, in the form of Cartesian coordinates or another suitable coordinate system.

In various embodiments, the ligand fragment binding score includes one or more of mean binding affinity of the ligand fragment, median binding affinity of the ligand fragment, mode binding affinity of the ligand fragment, binding affinity of the ligand, frequency with which the fragment occurs in the plurality of ligand fragments, calculated ADME (absorption, distribution, metabolism, and excretion) properties of the ligand fragment, and deviation between the mean ligand binding affinity corresponding to the ligand fragment-subregion combination and the overall mean binding affinity of the plurality of ligands.

In various embodiments, ligand fragments that appear more often than other ligand fragments are top lead fragments (TLF), each having a rank.

In various embodiments, the computational joining of two or more ligand fragments includes linking two or more TLFs for continuous subregions of the target protein. The linking is, in various embodiments, in the form of one or more bonds between any non-hydrogen atoms in the ligand fragments.

In various embodiments, the computational joining includes linking two or more TLFs such that the sum of the ranks of the TLFs is an integer from 3 to 10.

In various embodiments the selection of fragments depends at least in part on a predicted subpocket location of the fragment.

In various embodiments, the selection of fragments depends at least in part on the amino acids which are computationally predicted to bind to the ligand fragment.

In various embodiments, a system configured to implement a computer-implemented method of drug design according to any of the methods described herein, the system including:
a computer system comprising one or more processors;
an optional display screen and an optional graphical user interface; and
the computer system being configured and adapted to implement deep reinforcement learning, the computer system being further configured and adapted to be provided with objectives for a therapeutically effective drug, wherein the one or more processors are configured to execute a set of instructions that:
(a) provide a computer model of an atomic structure of a target protein and a plurality of ligands independently docked in a binding region in the target protein, wherein each ligand in the plurality of ligands has an associated binding affinity with the target protein;
(b) fragment each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally join two or more ligand fragments to form a drug, wherein a chemical structure of the drug is chemically stable and the two or more ligand fragments have fragment binding scores in the top 10% of a rank-ordered sorting of ligand binding scores of the plurality of ligand fragments. In various embodiments, the two or more ligand fragments have fragment binding scores in the top 1, 2, 3, 4, 5, 6, 7, 8, 9, 10, 11, 12, 13, 14, or 15% of a rank-ordered sorting of ligand binding scores of the plurality of ligand fragments.

In various embodiments, the computer system is further configured and adapted to achieve a high binding affinity with the protein target.

In various embodiments, the computer system is further configured and adapted to achieve drugs which are highly soluble and minimally hydrophobic.

In various embodiments, the computer system is further configured and adapted to learn how to optimize drug design by automatically reinforcing designs that better meet objectives in a deep reinforcement learning training process.

In various embodiments, a computer-readable recording medium storing instructions to execute any of the methods described herein.

In various embodiments, a computer-implemented method of drug design is provided, the method including:
(a) accessing a computer model of a target protein and computer models of a plurality of ligand structures;
(b) obtaining a predicted binding affinity between each of the ligands and the target protein;
(c) providing protein-ligand bond profiling data based on computational predictions on how each of the plurality of ligand structures binds with the target protein;
(d) fragmenting the plurality of ligand structures into computer models of ligand fragments; and
(e) providing scores for each of the ligand fragments using the predicted binding affinity and the protein-ligand bond profiling data.

In various embodiments, the method also includes a step (f) of providing a fragment database based on the scores for each of the ligand fragments.

In various embodiments, the method also includes a step (g) utilizing the resulting fragment database to design drug candidates in silico.

In various embodiments, the method also includes providing a computer system for implementing steps of (a)-(g), the computer system being configured and adapted to implement a deep reinforcement learning (Deep RL) training process.

In various embodiments, the method also includes providing objectives to the computer system, the objectives being defined for a drug or lead compound with desirable drug-like properties.

In various embodiments, the objectives for any of the methods or systems described herein are selected from the group consisting of high binding affinity with the protein target, high aqueous solubility, and low lipophilicity. In various embodiments, the desired objectives for drug properties for drug candidates or lead compounds identified by any of the methods or systems described herein include one or more of:
i) a molecular weight of the drug or desired drug of about 200 to about 600 g/mol;
ii) a lipophilicity as measured by the octanol-water partition coefficient (A log P or C log P) of about-2 to about 6;
iii) 4 or fewer hydrogen bond donors;
iv) 2 to 10 hydrogen bond acceptors;
v) a polar surface area of about 25 to about 200 Å²;
vi) 10 or fewer rotatable bonds; and
vii) 4 or fewer aromatic rings.

The drug candidates and/or lead compounds identified by the methods described herein, in various embodiments, have an in vitro or in vivo potency (as measured by IC₅₀ or EC₅₀) against its target receptor of less than, at least, or equal to about 20, 15, 10, 9, 8, 7, 6, 5, 4, 3, 2 or about 1 μM. The drug candidates and/or lead compounds identified by the methods described herein, in various embodiments, has an in vitro or in vivo potency (as measured by IC₅₀ or EC₅₀) against its target receptor of less than, at least, or equal to about 900, 800, 700, 600, 500, 400, 300, 200, 100, 90, 80, 70, 60, 50, 40, 30 20, 10, 9, 8, 7, 6, 5, 4, 3, 2, 1, 0.5, 0.1, 0.05, or about 0.01 nM.

In various embodiments, the computer system is configured and adapted to learn the best assemblies of fragments to formulate lead compounds that meet the provided objectives, as described herein.

In various embodiments, the computer system is configured and adapted to learn how to optimize drug design or lead compound design by automatically reinforcing designs (chemical structures) that better meet the provided objectives in the Deep RL training process.

In various embodiments, a computer-implemented method of drug design includes the steps of:
(a) accessing a computer model of an atomic structure of a target protein and a plurality of ligands independently docked in a binding region in the target protein, wherein each ligand in the plurality of ligands has an associated binding affinity with the target protein;
(b) fragmenting each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally joining two or more ligand fragments to form a drug, wherein a chemical structure of the drug is chemically stable and the two or more ligand fragments have fragment binding scores above a certain threshold of a rank-ordered sorting of ligand binding scores of the plurality of ligand fragments.

In various embodiments, the certain threshold is set at the 70th, 75th, 80th, 85th, 90th, 95th, or 99th percentile.

In various embodiments, a computer-implemented method of drug design includes the steps of:
(a) accessing a computer model of an atomic structure of a target protein and a plurality of ligands independently docked in a binding region in the target protein, wherein each ligand in the plurality of ligands has an associated binding affinity with the target protein;
(b) fragmenting each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally joining two or more ligand fragments to form a drug, wherein a chemical structure of the drug is chemically stable and the two or more ligand fragments have fragment binding scores in the top of a certain percentage of a rank-ordered sorting of ligand binding scores of the plurality of ligand fragments,
wherein the certain percentage is dependent on a number of computationally combined compounds.

Although specific software packages are described herein, the present methods and systems are not limited to being implemented with any particular combination of software, and any suitable software suite (e.g., from Schrodinger) or combination of software can be used.

### Database Creation FDSL-DD Overview

The input to the FDSL-DD can be the output files of ligand screening (e.g., from AutoDock Vina). The output files can include PDBQT (Protein Data Bank, Partial Charge (Q), & Atom Type (T)) format representation of a ligand structure in its predicted docking conformations with the protein, along with predicted binding affinities. The minimum binding affinity solution (or threshold) can be selected (or provided). The ligand PDBQT and protein PDB files can be merged and then fed into Protein-Ligand Interaction Profiler (PLIP) which predicts ligand atom-amino acid bonds. The ligand PDBQT files can also converted into a simplified molecular-input line-entry system (SMILES) representation, which can then be fed into a computational fragmentation tool (e.g., breaking of retrosynthetically interesting chemical substructures (BRICS) or retrosynthetic combinatorial analysis procedure (RECAP). The fragments from each ligand can then be collected. The FDSL-DD can then output a database or table of fragments (e.g., with SMILES representation) at different locations in the binding pocket determined by the amino acids to which the fragments bind in a parent ligand. There can be more than one entry for a particular fragment if it is found to bind in different locations in different fragments.

To demonstrate the potential of the created fragment library or pipeline, three very different protein targets have been chosen as examples in a study, in accordance with exemplary embodiments of the present disclosure. The first, tumor necrosis factor alpha induced protein 8-like 2 (TIPE2), is a transport protein that can induce leukocyte polarization, sustaining chronic inflammation and ultimately supporting tumorigenesis. Inhibition of TIPE2 would provide a therapeutic option for solid tumor cancers. The second, RelA, a protein that detects amino acid starvation activating the stringent response in bacteria which leads to persister cell formation. Persister cells can withstand 1000 times the antibiotic concentrations of their normal cell counterparts, so inhibit RelA and antibiotics can be used to eradicate the bacteria, and mostly importantly bacterial biofilms. The final protein utilized in this study is the receptor binding domain (RBD) of the S1 subunit of the spike protein (S-protein) of severe acute respiratory syndrome coronavirus 2 (SARS-CoV-2). The S-protein RBD binds to human angiotensin-converting enzyme (ACE-2), facilitating viral entry. The choice of the first two proteins was because no strong inhibitors have been reported for those two proteins. The choice of the last protein was due to the need of medicinal treatment for the pandemics caused by SARS-CoV-2.

### Methods of an Exemplary Study

The methods and study described herein are exemplary in nature. The examples are illustrative and the embodiments of the study are exemplary in nature and are not intended to limit the invention to the embodiments and examples described.

### Preparation of Receptor and Ligands

As an example, the two-stage computational drug design methodology described herein is performed on three distinct protein targets found in different kinds of organisms, i.e., human, bacterial, and viral, and which are in turn implicated in very different kinds of diseases and contexts. Tumor necrosis factor-alpha-induced protein-like (TIPE2), a transport protein that can induce leukocyte polarization, sustaining chronic inflammation and ultimately supporting solid cancer tumorigenesis; TIPE2 inhibition would provide a therapeutic option for solid tumor cancers. Bacterial protein RelA, which plays a role in detecting amino acid starvation, activating a stringent response in bacteria that leads to persister cell formation.

Persister cells can withstand upwards of 1000 times the antibiotic concentrations of their normal cell counterparts; accordingly, inhibiting RelA can allow antibiotics to eradicate bacteria in biofilms. The receptor binding domain (RBD) of the S1 subunit of the spike protein (S-protein) of severe acute respiratory syndrome coronavirus 2 (SARS-CoV-2) and the SARS-CoV-2 spike protein receptor binding domain (RBD), which binds to human angiotensin-converting enzyme (ACE-2), thereby facilitating viral entry and representing potential target for antiviral therapeutics for COVID-19. The computational studies herein demonstrate the potential of the methods to significantly enhance the efficiency and effectiveness of computational drug design.

In accordance with a study described herein, the crystal structures of the protein files (e.g., TIPE2 (PDB ID: 3F4M), RelA (PDB ID: 5IQR), and S-protein (PDB ID: 6M0J) were retrieved from the RCSB Protein Data Bank. The structures were cleaned (removing all waters, co-crystalized proteins, and co-crystalized atoms) and prepared in AutoDockTools-1.5.6 with the addition of polar hydrogens and calculation of Gasteiger charges. A plurality of ligands were taken from a “drug-like” library (e.g., from an Enamine Ltd.) consisting of around 250,000 molecules. The plurality of ligands were retrieved and optimized using OpenBabel through the generation of 3D structures, addition of charges, and minimization using the MMFF94 force field.

### Grid Box and Molecular Docking

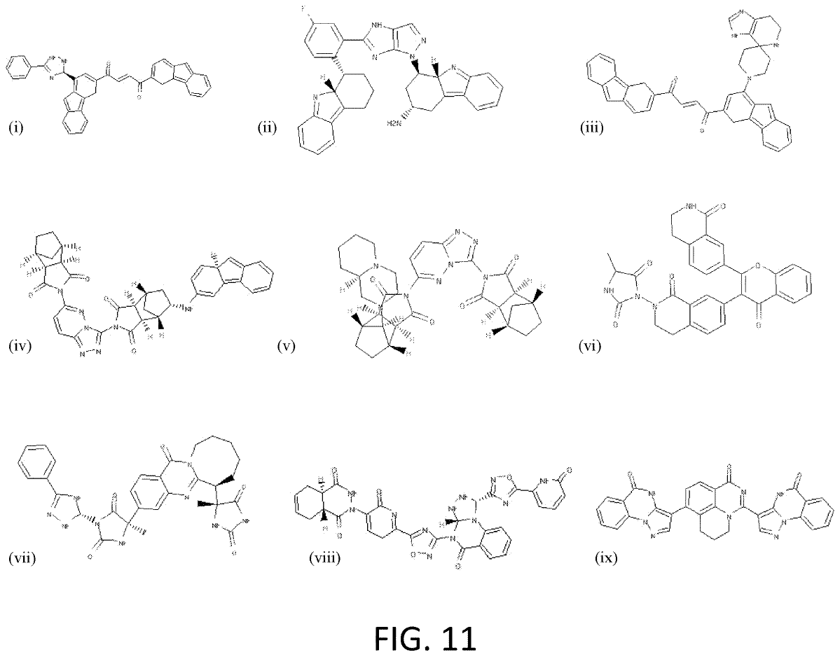

Grid boxes were generated for each protein in accordance with their binding site. The grid box for RelA was centered at x=297.894, y=163.593, and z=219.301 with dimensions of 25.000 Å. For the S-protein the box of dimensions 22.000 Å×42.000 Å×22.000 Å were centered at x=−27.878, y=25.205, and z=5.514. Due to large binding cavity of TIPE2, 4 grid boxes were generated. These grid boxes span the pocket entrance, occluding the cavity. All grid box quadrants are of 12.000 Å dimensions with: quadrant 1 centered at x=60.677, y=5.646, and z=17.000; quadrant 2 centered at x=62.636, y=11.365, and z=19.959; quadrant 3 centered at x=68.067, y=10.738, and z=18.594; and quadrant 4 centered at x=67.024, y=5.362, and z=17.113. All ligands were docked with each protein using their respective grid boxes. High throughput molecular docking calculations of mass libraries were performed using AutoDock Vina 1.1.2. on a high-performance computer cluster (called “Picotte”). Docking with TIPE2 generated four times the output, as every ligand was docked in each quadrant grid box. FIG. 11 shows a schematic of the prescreening workflow.

### FDSL-DD

The AutoDock Vina output file can include nine binding solutions, each with a predicted protein-ligand binding affinity. The FDSL-DD can select the lowest binding affinity solution, and it can extract the PDBQT file and binding affinity value. The ligand PDBQT-format file can be converted to a PDB format and merged with the protein PDB file (e.g., using the VINA command line merge tool). This provides the input for the Protein Ligand Interaction Profiler (PLIP). PLIP can perform a rule-based prediction of interactions between ligand atoms and protein amino acids, including the bond type and atom-residue pairs.

The PDBQT file of the ligand can also be converted to a MOL format file, or Molfile, which is a text file containing text information. The Molfile can then be broken into fragments using an algorithm designed to produce chemical fragments for drug design, generally by breaking the bonds in a chemically realistic manner, such as BRICS or RECAP. The fragmentation algorithm can be implemented in Python 3.8 using the RDKit open source chemoinformatics software package (e.g., see http://www.rdkit.org). The fragments can then be associated with their “parent” ligands (i.e., the ligands that were fragmented). Many ligands can have multiple parent ligands (i.e., they will have appeared as the fragments of multiple ligands).

The location of the fragment in the binding pocket can then be identified for each parent ligand (e.g., following the procedure implemented by Tang et al., 2014). The ligand atoms constituting the fragment can be identified by finding the maximum common substructure (MCS) between the fragment and ligand (e.g., by using RDKit). The XML-formatted output of PLIP can be parsed to obtain the residues identified as binding to the ligand atoms corresponding to the fragment. The fragment can then be associated with those residues. In many applications, groups of protein residues can be defined as binding pocket subregions (e.g., subregions A, B, or C). If a fragment's constituent atoms bind to the residues in a subregion, then the fragment can be associated with that subregion. In some ligand contexts, a fragment will bind to residues in multiple subregions.

As illustrated in FIG. 2, a table or database can then be created with entries for each distinct fragment-subregion combination. The fragment can be stored in a SMILES format, which can be a string that includes the structural information required to reconstruct the ligand. The fragment can have multiple entries if it is found binding to multiple binding pocket subregions in different ligand contexts. For each fragment-subregion combination, binding affinity statistics can also be computed. Generally, the database will include the mean binding affinity predicted (e.g., by AutoDock Vina) for all parent ligands including that fragment-subregion combination. It may also (or instead) include the median or mode binding affinity. Another statistic that can be calculated is the deviation between the mean parent ligand binding affinity for that fragment-subregion combination and the overall mean binding affinity for all ligands in the screening experiment. Finally, the database can also include the number of parent ligands (“Count” in FIG. 2) in which the fragment-subregion combination is found.

### Computational Ligand Design

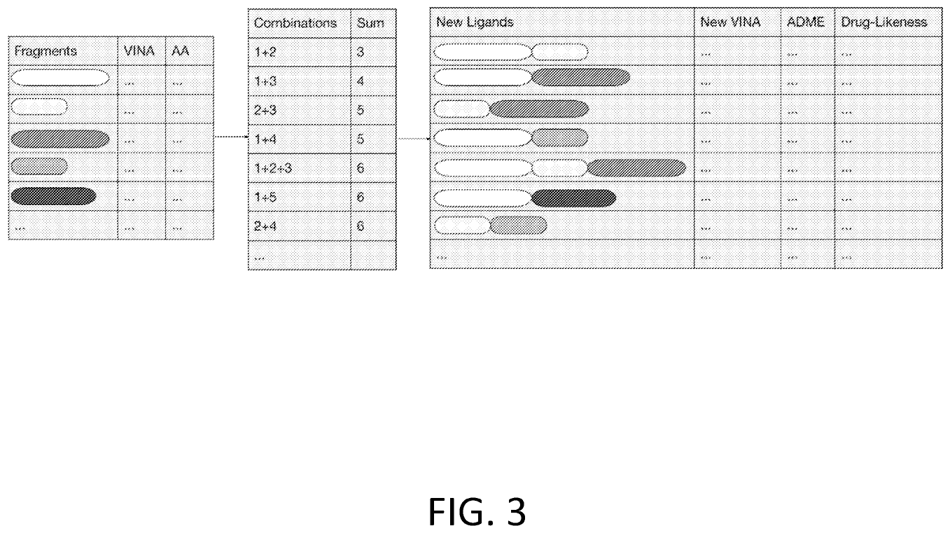

A basic prioritized search for ligands based on the fragment library generated by the FLDD is described as follows and in FIG. 3. As an initial step, fragments are sorted based on the inferred binding affinity of their parent ligand, as illustrated in FIG. 2. Additional sorting can be performed; for example, fragments binding to particular regions (as illustrated in FIG. 1) may be placed in different pools, and fragments may be drawn from them according to the scheme illustrated in FIG. 3 and described here. A set of ligands is generated based on a triangular number sequence.

FIG. 3 illustrates a scheme used for generating fragments. The ranked fragment list is first created based on the Autodock VINA binding scores of their parent ligands and fragment counts as determined using the FLDD illustrated in FIG. 2. The search space is generated by using triangular numbers to generate all possible combinations of ranked fragments up to a given threshold. The example illustrated in FIG. 3 is to generate all possible ranks up to rank 4, which is based on the triangular number 6, which includes all possible ways of summing to 3, 4, 5, and 6. These are then the ranks of fragments that are used in combination. Possible candidate ligands are then generated from the prioritized rank list and evaluated using Autodock VINA, as well as for ADME and drug-likeness properties.

As illustrated in FIG. 3, all possible ways of summing the numbers m through n, where m is the first ranked fragment (generally m=1) and n is the last ranked considered for combination within the ranked list of fragments, are generated. For example, for m=1 and n=5, the combinations are 1+2, 1+3, 1+4, and 2+3. That means that candidate ligands are the combination of fragments ranked 1 and 2, 1 and 3, 1 and 4, and 2 and 3 respectively. Because most fragments are between 100-300 g/mol, combinations are only added with up to 5 fragments to comply with Lipinski's 500 g/mol rule. This significantly decreases computational burden, as only a subset of ligands that can be generated with 5 operands are evaluated.

BRICS fragments are combined using BRICS rules using the BRICS. Build command in RDKit. This is possible because the MolFile or SMARTS representations of BRICS fragments will store information about the broken bonds in BRICS fragmentation in isotopes. The BRICS.Build package in RDKit can utilize the isotope information to attempt to recombine fragments in new combinations according to the information in the isotopes. If the resulting molecule can be parsed by RDKit, then it is successful potential molecule. If isotopes remain, indicating potential binding sites, then other fragments can be added on to the growing ligand. If a different computational fragmentation procedure is used, then different computational methods can be used to assemble fragments. In various embodiments the methods provide for a computational pipeline that allows for RECAP fragments, if generated, to be combined through text processing of SMILES strings by replacing the (*) wildcard character used to indicate broken bonds (i.e. open binding sites).

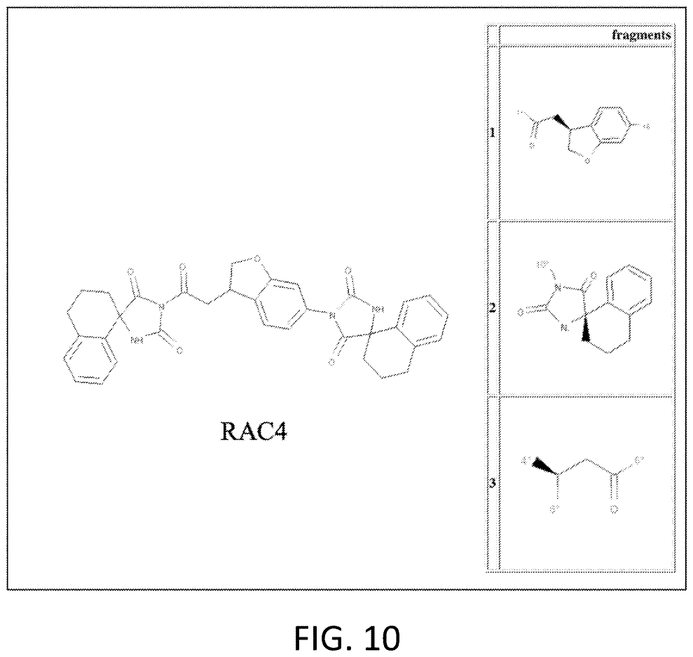

It is important to note that not every fragment fed into the BRICS recomposition algorithm is guaranteed to be included in the final molecule. FIG. 10 shows, for example, RAC4 and its parent fragments. As shown in FIG. 10, in the case of RAC4, the third fragment fed did not end up in the final ligand. Additionally, BRICS may repeat fragments to fill the valences of each incorporated fragment. In the case of RAC4, fragment 1 and 3 had more than one fragment end. Regardless of which fragment was used, at least one repeat of fragment 2 is necessary to fill in all valences since fragment 2 is the only fragment with only one exposed end. Although repeat fragments are not ideal from a diversity perspective, in some cases, they enable molecules with better binding affinities to be generated, like in the case of RAC4.

The resulting ligands are then evaluated based on their binding affinity using Autodock VINA. Optionally, in silico absorption, distribution, metabolism, and excretion (ADME) properties are also calculated using SwissADME, as well as other drug likeness properties such as the Lipinski's rule of 5 parameters, using built-in RDKit features in the rdkit. Chem.Lipinski module. The highest-ranking ligands on the basis of binding affinities and ADME properties are also visualized in protein-ligand complexes with PyMOL and ChimeraX-1.2.1.

### Results

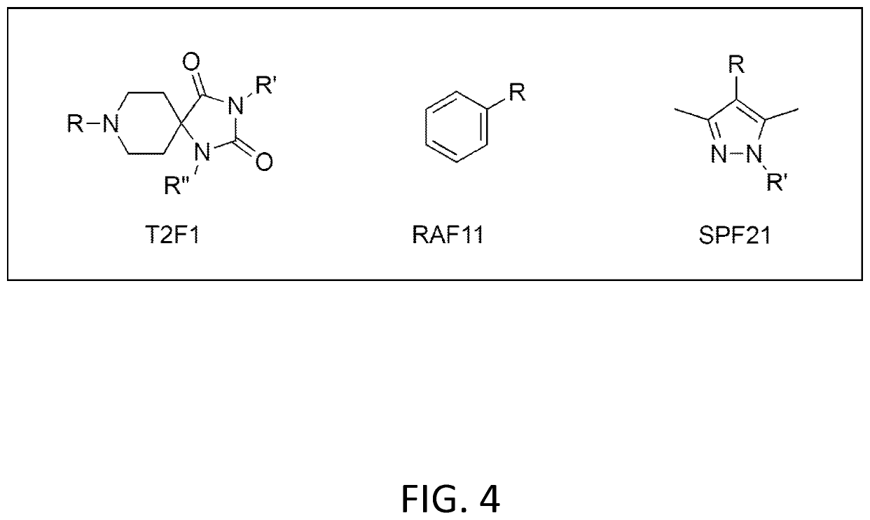

Fragment databases were created for each protein. Table 1 shows the most frequently occurring fragments in the top 10% of highest binding ligands for each protein. For TIPE2, 1,3,8-triazaspiro[4.5]decane-2,4-dione, or fragment T2F1 (see FIG. 4), appears 81 times from ligands with a mean binding affinity of −7.72 kcalmol⁻¹, and possible connections at the 1, 3, and 8 position of TIPE2. For RelA, benzene is the most frequently occurring fragment, with a count of 584 in ligands with a mean binding affinity of −8.33 kcalmol⁻¹ at certain positions. For the S protein, 3,5-dimethyl-1H-pyrazole, or SPF21, was the most frequent fragment, occurring 328 times with possible connection at positions 1 and 4.

TABLE 1

| Fragment SMILES | Protein | Fragment | Domain | Mean | Mode | Median | Count |
|---|---|---|---|---|---|---|---|
| [5*]N1CCC2(CC1)C(═O) | TIPE2 | T2F1 | A44 | −7.72 | −7.90 | −7.70 | 81 |
| N([10*])C(═O)N2[10*] | | | | | | | |
| [10*]N1C(═O)[N]C([13*]) | RelA | RAF11 | N187, | −8.33 | −8.00 | −8.30 | 584 |
| ([13*])C1═O | | | K195, | | | | |
| | | | Y319 | | | | |
| [5*]N1CCc2nnc([14*])n2CC1 | Spike | SPF21 | F497, | −7.22 | −7.00 | −7.10 | 328 |
| | | | Y505 | | | | |

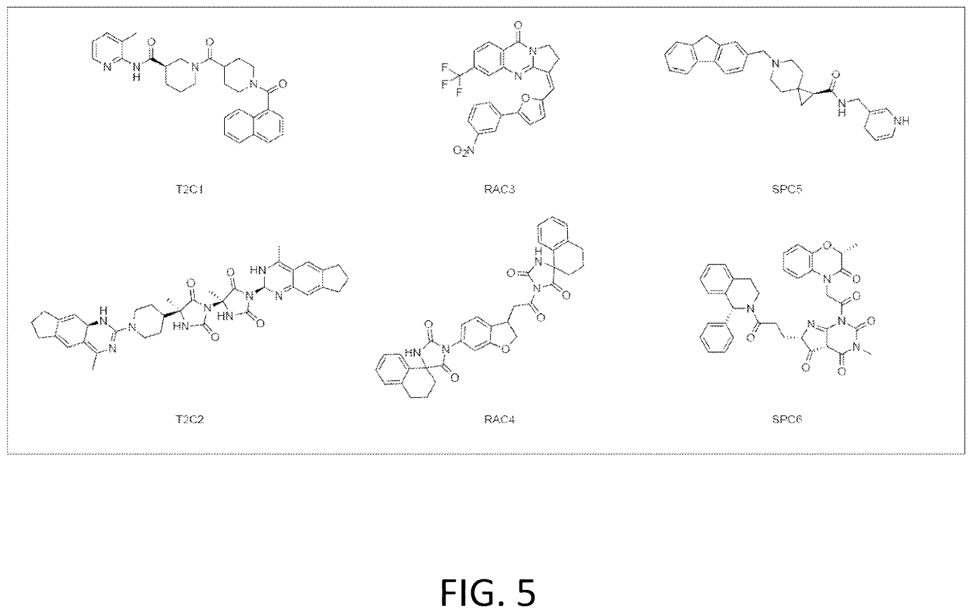

From these generated fragment libraries superior binding ligands were constructed for each protein, the highest binding pictured in FIG. 5. For comparison, Table 2 shows the highest binding ligands from the high throughput virtual screening (HTVS) of the Enamine library (or database) of 8 million compounds (of a total of 210 million Enamine compounds) with each protein against the highest binding ligand produced from FLDD. Some predicted ADME properties of the compounds that are utilized in the training environment have also been noted. An increase in binding affinity is seen for each constructed ligand.

Table 2 illustrates a comparison of top binding ligand from high throughput virtual screening (HTVS) and top binding ligand from FLDD for each protein target. Included are binding affinities from high throughput method and from the reinforced learning FLDD and computed ADME properties.

TABLE 2

| | TIPE2 | | RelA | | S-Protein | |
|---|---|---|---|---|---|---|
| Method | HTS | FLDD | HTS | FLDD | HTS | FLDD |
| Ligand | T2C1 | T2C2 | RAC3 | RAC4 | SPC5 | SPC6 |
| Name | | | | | | |
| Binding | −9.8 | −13.4 | −10.2 | −11.9 | −9.1 | −12.0 |
| Affinity | | | | | | |
| (kcalmol⁻¹) | | | | | | |
| Molecular | 484.59 | 484.59 | 484.59 | 484.59 | 484.59 | 484.59 |
| Weight | | | | | | |
| (gmol⁻¹) | | | | | | |
| Solubility | −4.94 | −4.63 | −5.68 | −5.44 | −4.72 | −5.63 |
| (ESOL) | | | | | | |
| Partition | 2.91 | 2.8 | 2.78 | 3.25 | 3.59 | 2.08 |
| Coefficient | | | | | | |
| (MlogP) | | | | | | |
| TPSA | 82.61 | 150.84 | 93.85 | 182.07 | 44.37 | 136.97 |
| Log Kₚ | −6.74 | −9.55 | −5.88 | −8.03 | −6.25 | −7.96 |
| (cm/s) | | | | | | |
| GI | High | Low | Low | Low | High | High |
| Absorption | | | | | | |
| BBB | No | No | No | No | Yes | No |
| permeant | | | | | | |

Tables 3A and 3B illustrates a comparison of drug-likeness according to 5 widely accepted guidelines. The Lipinski filter: MW ≤500, M Log P ≤4.15, hydrogen bond acceptors ≤10, and hydrogen bond donors ≤5. The Ghose filter: 160≤MW ≤480, −0.4≤W Log P ≤5.6, 40≤MR ≤130, 20≤atoms ≤70. The Veber filter: rotatable bonds ≤10 and TPSA ≤140. The Egan filter: W Log P ≤5.88 and TPSA ≤131.6. The Muegge filter: 200≤MW ≤600, −2≤X Log P ≤5, TPSA ≤150, number of rings ≤7, number of carbons >4, number of heteroatoms >1, number of rotatable bonds ≤15, hydrogen bond acceptors ≤10, and hydrogen bond donors ≤5.

TABLE 3A

| | TIPE2 | | RelA | | S-Protein | |
|---|---|---|---|---|---|---|
| Method | HTVS | FLDD | HTVS | FLDD | HTVS | FLDD |
| Ligand | T2C1 | T2C2 | RAC3 | RAC4 | SPC5 | SPC6 |
| Name | | | | | | |
| Binding | −9.8 | −13.4 | −10.2 | −12.4 | −9.1 | −12.0 |
| Affinity | | | | | | |
| (kcalmol⁻¹) | | | | | | |
| Molecular | 484.59 | 673.76 | 453.37 | 590.63 | 426.57 | 647.69 |
| Weight | | | | | | |
| (gmol⁻¹) | | | | | | |
| Solubility | −4.94 | −4.63 | −5.68 | −5.84 | −4.72 | −5.63 |
| (ESOL) | | | | | | |
| Partition | 2.91 | 2.8 | 2.78 | 2.97 | 3.59 | 2.08 |
| Coefficient | | | | | | |
| (MlogP) | | | | | | |
| TPSA | 82.61 | 150.84 | 93.85 | 125.12 | 44.37 | 136.97 |
| Log Kₚ | −6.74 | −9.55 | −5.88 | −7.31 | −6.25 | −7.96 |
| (cm/s) | | | | | | |
| GI | High | Low | Low | High | High | High |
| Absorption | | | | | | |
| BBB | No | No | No | No | Yes | No |
| permeant | | | | | | |

Table 3A shows a comparison of top binding ligands from high throughput virtual screening (HTVS) and top binding ligand from FDSL-DD for each protein target. Included are binding affinities from high throughput method and from the FDSL-DD and computed ADME properties.

TABLE 3B

| | TIPE2 | | RelA | | S-Protein | |
|---|---|---|---|---|---|---|
| | | FDSL- | | FDSL- | | FDSL- |
| Method | HTvS | DD | HTvS | DD | HTvS | DD |
| Ligand Name | T2C1 | T2C2 | RAC3 | RAC4 | SPC5 | SPC6 |
| Lipinski | Yes | No | Yes | Yes | Yes | No |
| Ghose | No | No | No | No | No | No |
| Veber | Yes | No | Yes | Yes | Yes | Yes |
| Egan | Yes | No | No | Yes | Yes | No |
| Muegge | Yes | No | Yes | No | Yes | No |

Table 1B. Comparison of drug-likeness. The Lipinski filter; MW ≤500, M Log P ≤4.15. The Ghose filter; 160≤MW ≤480, −0.4≤W Log P ≤5.6, 40≤MR ≤130, 20≤atoms ≤70. The Veber filter; TPSA ≤140. And the Egan filter; W Log P ≤5.88 and TPSA ≤131.6. The Muegge filter; 200≤MW ≤600, −2≤X Log P ≤5, TPSA ≤150.

Table 3A shows the highest binding ligands from the HTVS of the Enamine library with each protein against the highest binding ligand produced from the FDSL-DD. Select predicted ADME properties that are utilized in the training environment have also been noted. An increase in binding affinity is seen for each constructed ligand. The most substantial increase was exhibited by the ligand designed for TIPE2, with a 3.6 kcal mol⁻¹ difference between the top ligand from the FDSL-DD and top ligand from the HTVS. Solubility and partition coefficient remained within the same range of the HTVS ligands. The molecular weight saw a significant increase for T2C2 and SPC6. And while approved drugs have been increasing in molecular weight and surpassing the 500 g mol⁻¹ maximum guideline in recent years bringing this value down with future adjustments could also contribute to improving solubility. The FDSL-DD synthesized ligand, RAC4, is the best overall in terms of drug-likeness (Table 3B), meeting the constraints of three widely accepted guidelines; Linpinski, Veber and Egan. Imposing stricter penalties into the selection method will promote better candidates, such as RAC4, that not only exhibit satisfactory binding affinities but desirable ADME and pharmacokinetic properties.

Table 4 lists the ten most frequently occurring fragments in the top 10% of highest binding ligands for TIPE2.

TABLE 4

| Fragment SMILES | Fragment | Domain | Mean | Mode | Median | Count |
|---|---|---|---|---|---|---|
| [5*]N1CCC2(CC1)C(═O)N([10*])C(═O)N2[10*] | T2F1 | A44 | −7.72 | −7.90 | −7.70 | 81 |
| [10*]N1C(═O)[N]C([13*])([13*])C1═O | T2F2 | F67 | −7.76 | −7.60 | −7.80 | 74 |
| [5*]N1CCc2nnc([14*])n2CC1 | T2F3 | L161 | −8.04 | −8.20 | −8.10 | 63 |
| [15*]C1Cc2ccccc2C1 | T2F4 | L41, | −8.01 | −8.40 | −7.90 | 61 |
| | | I45, I8, | | | | |
| | | L16 | | | | |
| [5*]N1CCc2nnc([14*])n2CC1 | T2F5 | S12 | −7.79 | −7.00 | −7.70 | 60 |
| [5*]N1CCc2nnc([14*])n2CC1 | T2F6 | L16 | −7.73 | −7.90 | −7.65 | 56 |
| [8*]C(F)(F)F | T2F7 | L40, | −7.71 | −7.40 | −7.60 | 55 |
| | | F67 | | | | |
| [10*]N1C(═O)[N]C([13*])([13*])C1═O | T2F8 | A44 | −7.70 | −8.10 | −7.90 | 50 |
| [16*]c1cccc2ccccc12 | T2F9 | A44, | −7.65 | −8.10 | −7.55 | 50 |
| | | L40, | | | | |
| | | L71, | | | | |
| | | F124, | | | | |
| | | V123, | | | | |
| | | V43 | | | | |
| [16*]c1ccccc1 | T2F10 | L40, | −7.63 | −8.40 | −7.65 | 46 |
| | | V123 | | | | |

Table 5 lists the ten most frequently occurring fragments in the top 10% of highest binding ligands for RelA

TABLE 5

| Fragment SMILES | Fragment | Domain | Mean | Mode | Median | Count |
|---|---|---|---|---|---|---|
| [16*]c1cccc2ccccc12 | RAF11 | N187, | −8.33 | −8.00 | −8.30 | 584 |
| | | K195, | | | | |
| | | Y319 | | | | |
| [1*]C(═O)C1═C2CC═CC═C2Oc2ccccc21 | RAF12 | A347, | −8.42 | −8.30 | −8.40 | 90 |
| | | Y319 | | | | |
| [12*]S(═O)(═O)c1ccc([16*])cc1 | RAF13 | R61, | −8.33 | −8.20 | −8.25 | 72 |
| | | E348, | | | | |
| | | K254 | | | | |
| [16*]c1ccc2c(c1)OCO2 | RAF14 | K262 | −8.24 | −8.90 | −8.35 | 72 |
| [5*]N1CC(═O)[N]c2ccccc21 | RAF15 | A347, | −8.36 | −8.70 | −8.60 | 68 |
| | | Y19 | | | | |
| [14*]c1nc2ccccc2s1 | RAF16 | H323, | −8.27 | −8.90 | −8.25 | 68 |
| | | Y310 | | | | |
| [16*]c1ccccc1C | RAF17 | K254, | −8.23 | −8.10 | −8.10 | 66 |
| | | K262, | | | | |
| | | S258 | | | | |
| [16*]c1ccc2c(C)cc(═O)oc2c1 | RAF18 | K254 | −8.39 | −8.60 | −8.50 | 64 |
| [16*]c1ccc2c(c1)OCCO2 | RAF19 | K254 | −8.20 | −8.60 | −8.30 | 62 |
| [16*]c1ccccc1 | RAF20 | E335, | −8.22 | −9.00 | −8.30 | 60 |
| | | T333, | | | | |
| | | V325 | | | | |

Table 6 lists the ten most frequently occurring fragments in the top 10% of highest binding ligands for S-Protein

TABLE 6

| Fragment SMILES | Fragment | Domain | Mean | Mode | Median | Count |
|---|---|---|---|---|---|---|
| [9*]n1nc(C)c([16*])c1C | SPF21 | F497, | −7.22 | −7.00 | −7.10 | 328 |
| | | Y505 | | | | |
| [12*]S(═O)(═O)c1ccc2ccccc2c1 | SPF22 | N501, | −7.34 | −7.50 | −7.40 | 180 |
| | | Y505 | | | | |
| [14*]c1nc2ccccc2s1 | SPF23 | D405, | −7.25 | −7.40 | −7.30 | 138 |
| | | E406 | | | | |
| [16*]c1ccc2c(c1)OCCO2 | SPF24 | R403, | −7.24 | −7.00 | −7.25 | 132 |
| | | Q409 | | | | |
| [16*]c1ccc2c(c1)OCCO2 | SPF25 | R403, | −7.23 | −7.40 | −7.30 | 132 |
| | | Q409, | | | | |
| | | K417, | | | | |
| | | Y453 | | | | |
| [12*]S(═O)(═O)c1ccc2ccccc2c1 | SPF26 | N501, | −7.28 | −7.40 | −7.40 | 120 |
| | | G496, | | | | |
| | | Y505 | | | | |
| [5*]N1c2cccc3cccc(c23)S1(═O)═O | SPF27 | R403, | −7.45 | −7.50 | −7.40 | 118 |
| | | Y495, | | | | |
| | | Y505 | | | | |
| [5*]N1c2cccc3cccc(c23)S1(═O)═O | SPF28 | R403, | −7.40 | −7.50 | −7.40 | 110 |
| | | F497, | | | | |
| | | Y495, | | | | |
| | | Y505 | | | | |
| [12*]S(═O)(═O)c1ccc2c(c1)CCCC2 | SPF29 | N501, | −7.32 | −7.50 | −7.35 | 108 |
| | | Y505 | | | | |
| [6*]C([N])═O | SPF30 | R403, | −7.23 | −7.10 | −7.10 | 100 |
| | | E406 | | | | |

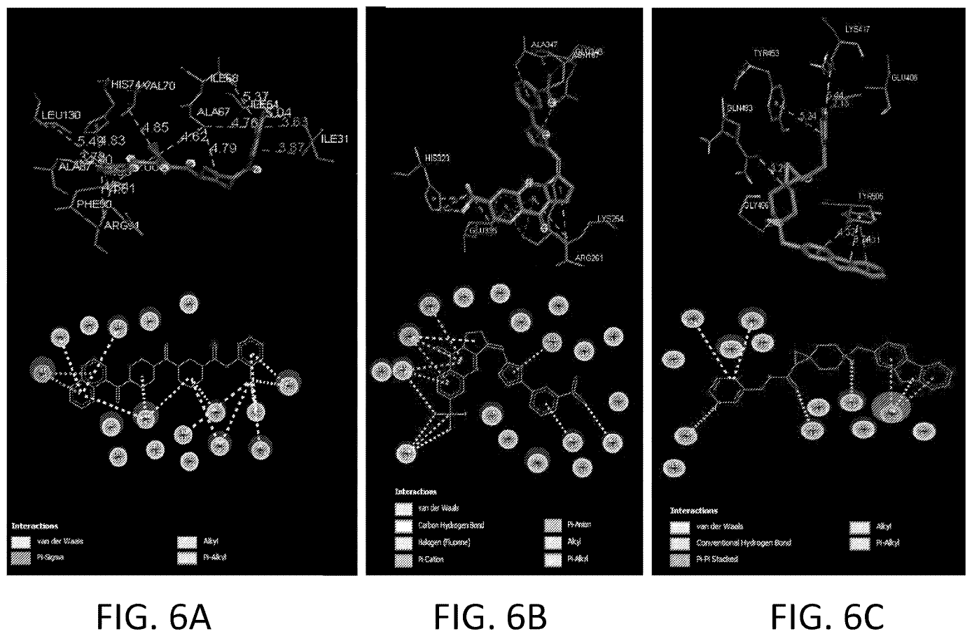

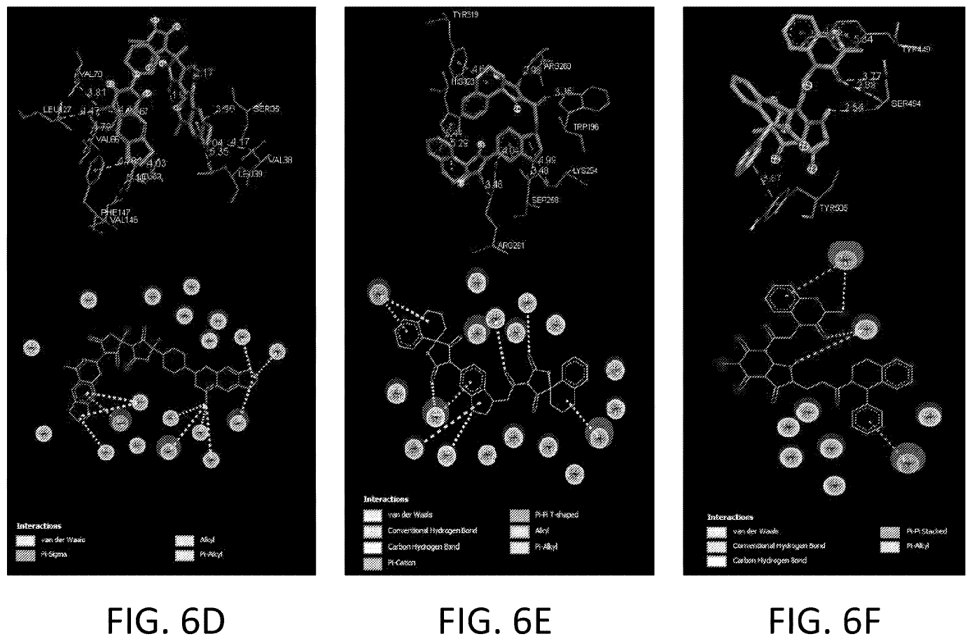

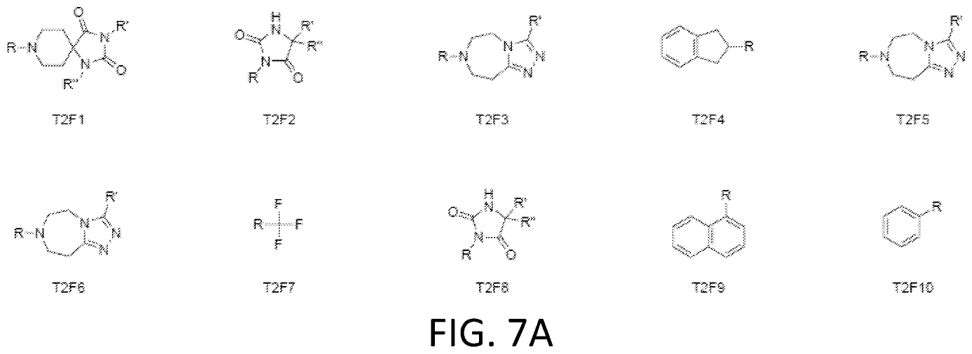

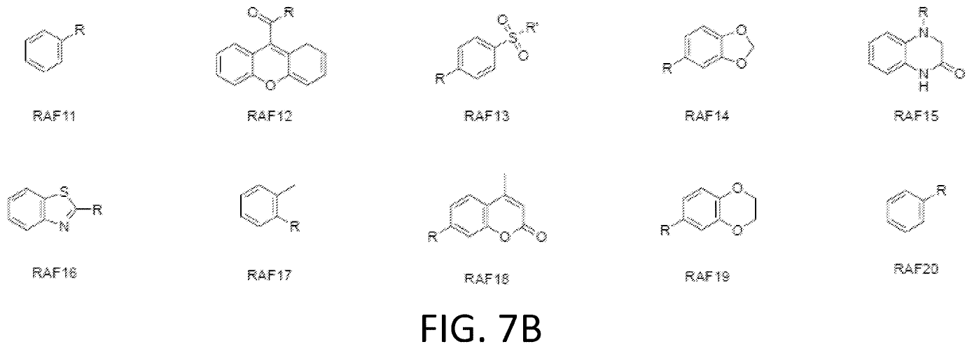

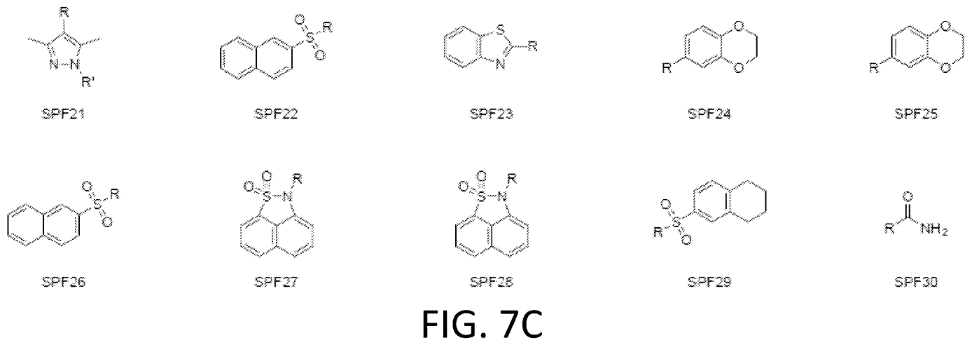

Referring to FIGS. 6A-6F, examination of the interactions of T2C1 versus T2C2 reveals both compounds interact with the amino acid residues of the binding pocket in the same way; alkyl-alkyl, π-alkyl, π-sigma and van der Waals interactions. The difference between these two compounds is size. T2C2 is nearly 200 g mol⁻¹ larger than T2C1 with an additional 8 Å in length. As such the significant increase in binding affinity is seemingly an increased surface area.

Between RAC3 and RAC4 there is a 2.4 kcal mol⁻¹ increase in affinity for the FDSL-DD constructed ligand. This stronger binding potential is exhibited in the addition of hydrogen bonding and π-π stacking for RAC4. The remainder of the interactions between RelA and RAC4 are similar to the interactions between RelA and RAC3; van der Waals, carbon-hydrogen, π-cation, alkyl, and π-alkyl interactions contributing to the binding affinity for both RAC4 and RAC3 were all observed.

The potential inhibitors for the Spike protein, SPC5 and SPC6, both exhibit van der Walls, hydrogen bonding, π-π stacking and π-alkyl interactions. The built ligand, SPC6, also includes carbon-hydrogen bond interactions and is significantly larger in size, contributing to the increase in binding affinity.

### Genetic Algorithm (Phase 1 of Fragment Synthesis)

The genetic algorithm requires the target receptor's PDB (Protein Data Bank) file, an Autodock VINA configuration file (as described in the previous subsection for ligand prescreening), and the output of the fragmentation and analysis pipeline described in the preceding subsection, as shown in FIG. 2. The resulting table is sorted based on the binding affinity to the receptor without considering target residues (i.e. the overall median binding affinity for each fragment). In the current version of the code, BRICS fragments are required, although some modification can make it compatible with any fragmentation protocol.

### Genetic Algorithm Overview

An individual is defined as a collection of fragments used to generate a ligand. Each fragment within this collection is a gene and is represented by its unique index within the source table of library fragments. A rank weighting is calculated and assigned for each index within the source (parent) ligands. These are calculated by the index of the fragments within the input table, and a rank weighting function is described below:

$$Weight ( index ) = \frac{n - index}{n ( n + 1 )} \times 200 ,$$

where n is the length of the table (number of fragments). The weight index provides larger fractional weights to higher ranked fragments towards the top of the list, which are expected to produce ligands of a lower binding affinity because they were generated from ligands with lower binding affinity. These ranked weights are used to define a categorical distribution using the random.choices( ) function (a randomization function), which is used to select indexes when new fragments are being incorporated.

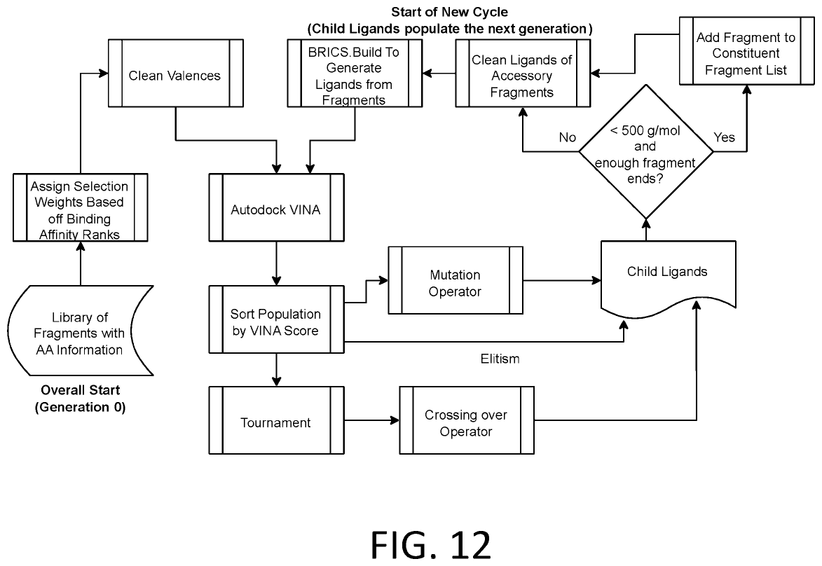

FIG. 12 shows an overview of the genetic algorithm procedure. To start, a random selection of fragments is chosen to seed the first generation. Hydrogens are added to unfilled valences (see Clean Valences block in FIG. 12), and they are run through Autodock VINA to evaluate them (see Autodock Vina block in FIG. 12). In addition, QED scores can be optionally calculated to determine drug likeness, and a combined QED and VINA score can be used for evaluating candidate ligands in the population instead of VINA score alone in the procedures described below.

The next generation is determined based off three operators: mutation, crossing over, and elitism. To start, the population is sorted by VINA score (the Sort Population by VINA Score block in FIG. 12), and the top ⅝th of the population are chosen to be ran through the mutation operator (the Mutation block in FIG. 12). These ligands, whether mutated or not, are added to the next generation.

Next, a tournament selection takes place (the Tournament block in FIG. 12), where each individual is compared against two other randomly chosen individuals, and the individual with the lowest binding affinity is chosen to be a parent used in the crossing over operator. Two unique individuals who won a tournament are run through the operator at a time, and the operator returns two children, which will both be included in the next generation. The crossing over operator accounts for ¼th of the following generation. The last ⅛th of the next generation is developed using elitism, where the individuals with the lowest binding affinity in the parent population are added without alteration.

The children to be used in the next generation are next screened to determine whether they have hit their max molecular weight of 500 g/mol. If not, or the number of fragment ends (unfilled valences) are an odd number, an additional fragment selected based off the rank weighting function described above is added to the ligand (the Add Fragment to Constituent Fragment List block in FIG. 12). If the maximum weight has been reached or the number of fragment ends are even, the ligands remain unaltered and are added to the fragment constituent list and progress to the Clean Ligands phase of the cycle.

To ensure that every fragment included in an individual is incorporated into the resultant ligand, each ligand is run through a cleaning function (the Clean Ligands of Accessory Fragments block in FIG. 12) to ensure there are enough fragments ends to accommodate all fragments. The BRICS.BUILD module in RDKit, which is used to generate each ligand from its constituent fragments, will not accommodate a fragment if there is not an end for it to bind to. Therefore, fragments which exceed the number of ends available to attach fragments are pruned, to avoid including it as a gene in future generations when it did not contribute to the evaluated structure. After running through the cleaning function, the BRICS.BUILD module generates ligands from the fragments (see BRICS.Build Generates Ligands from Fragments block in FIG. 12), and the children replace the parents as the new population. Then, the next generation begins.

### Genetic Algorithm Components

Mutation Operator: Based off the mutation rate supplied by the user, each fragment has a chance of mutating. In the event of a mutation, another fragment is substituted for the existing fragment, which is selected based off the categorical distribution calculated at the beginning using the random.choices( ) function. The ligands, whether mutated or not, are incorporated into the next generation.

Crossing Over Operator: The crossing over operator requires two parents to be inputted as well as a user-supplied crossing over rate. If a crossing over occurs, then a random index is selected between both individuals, and the fragments (indexes) between both individuals are swapped after that point. For example, assume an instance where two individuals with four corresponding fragments each are selected as parents:
Parent 1: [314, 132, 4813, 192]; Parent 2: [102, 8512, 591, 5123].

The indexes correspond to fragments in the original source CSV. If a crossing over event occurs, a random index is chosen as the index to perform the switch. Suppose the index 2 is selected. The following child ligands will be generated:
Child 1: [314, 132, 591, 5123]; Child 2: [102, 8512, 4831, 192].

These individuals will be incorporated into the next generation.

Generating Ligands from Fragments

The Build function from the BRICS package of RDKIT is used to generate ligands. The function is setup to only output complete SMILES, negating the need to manually add hydrogens to unfilled valences.

### Iterative Fragment Addition (Phase 2 of Fragment Synthesis)

The second stage of optimization is iterative fragment addition, which is effectively a hill-climbing algorithm for maximizing the drug design objective. In this study, we considered both binding affinity alone, as predicted by AutoDock VINA, as well as binding affinity in combination with the Quantitative Effectiveness of Drug-likeness (QED) score. The QED score was developed by Bickerton et al. as an improvement over rules, such as Lipinski's Rule of Five, which combine different thresholds and properties of ligands that tend to be associated with successful drugs. The QED score is a calculated by a formula based on a weighted sum of “desirability functions,” i.e., molecular properties associated with desirability for a particular class of drugs. As described herein, the default definition of QED from Bickerton et al., is utilized, including molecular weight (MW), octanol-water partition coefficient (ALOGP), number of hydrogen bond donors (HBD), number of hydrogen bond acceptors (HBA), molecular polar surface area (PSA), number of rotatable bonds (ROTB), the number of aromatic rings (AROM) and number of structural alerts (ALERTS). The QED score is computed using RDKit's qed( ) module (https://www.rdkit.org/docs/source/rdkit.Chem.QED.html).

The algorithm proceeds the same in either the QED+binding affinity or affinity-only, except that for QED+binding affinity, the optimization criterion is a sum of the two scores. The iterative fragment addition methodology requires a list of premade starter ligands and a dataset including fragments associated with amino acids of the target protein. The list of premade starter ligands is the output of the genetic algorithm, while the dataset of ligands and associated amino acids is the output of the initial prescreening and fragmentation pipeline. Before running the fragment addition, the fragment dataset is converted to a dictionary, where each amino acid is a key with at maximum 100 associated fragments based on the mean binding affinity of parent ligands.

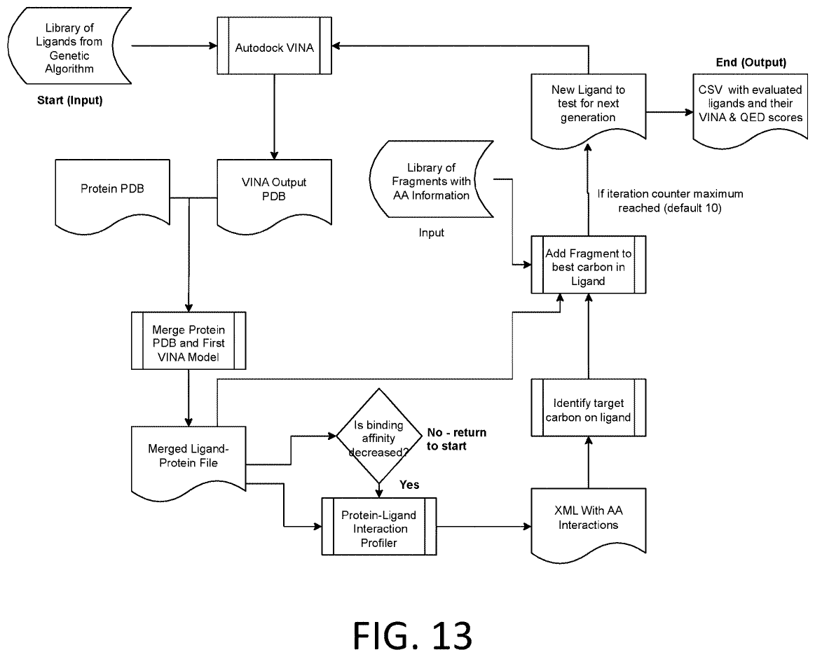

FIG. 13 shows an overview of the iterative fragment addition stage. At the start of each iteration, each ligand in the population is evaluated using Autodock VINA, using the same grid box and seeding as described above for the ligand prescreening stage. Next, the first model in the PDB output file of Autodock VINA is merged with the receptor protein PDB file. This step is in preparation for evaluation of the space using the Protein-Ligand Interaction Profiler (PLIP). Before running PLIP, each ligand in the population is compared against its predecessor to determine if the fragment addition was successful at decreasing binding affinity. If the binding affinity decreased, then the ligand is ready for another attempt at addition. If binding affinity increased, then the addition was unsuccessful at decreasing binding affinity, and the predecessor replaces the current ligand before the addition of a new fragment.

Next, all the merged ligand-protein PDB files with molecular weights less than 700 g/mol are ran through PLIP. Ligands with molecular weights greater than 700 g/mol are deemed too large for addition, as larger ligands take increasingly longer to evaluate using VINA and tend to make for worse drug targets. PLIP outputs an XML file containing information about relevant amino acids in the binding pocket of the protein. It also includes distances of the ligand to each of these amino acids. These distances are compared against distances between ligand carbons and protein carbons in the merged ligand-protein PDB, and the closest ligand carbon to an amino acid is selected as the target region to add a fragment.

After the target carbon is identified, the ligand and fragment are merged into one MOL object. A bond is formed between the target carbon in the ligand and the atom bound to a dummy atom (indicating a fragment end). All dummy atoms in the fragment are converted to hydrogens to fill the valence of the ligand. The new molecule is embedded into a 3D structure and MMFF94-optimized.

In the event RDKit is unable to optimize the newly generated ligand, the next best target carbon is selected, and another fragment addition is attempted. This is repeated multiple times until an optimizable ligand is generated, or the number of iteration attempts reaches ten. If the iteration counter limit is reached, the loop ends, and the SMILES of every ligand and associated VINA and QED score is written to a CSV file. If the iteration counter limit is not reached, the new ligands are evaluated using Autodock VINA and the cycle repeats.

Occasionally, RDKit will be able to embed and optimize the combined ligand and fragment MOL object but will be unable to embed and optimize the SMILES generated from the MOL object. For this reason, a filter is included every generation that tests to make sure that each ligand can be converted from its SMILES to an optimized 3D ligand. SMILES which cannot be converted will not be included in the final CSV file. This prevents inclusion of invalid ligands in the final output which cannot be converted to 3D MOL objects.

### Assessing Performance of Genetic and Iterative Optimization Ligand Designs Based on Prescreening Information

To determine the effect of fragment quality on the iterative algorithm results, four pools of fragments are created to be fed into the iterative algorithm for each protein target. Each pool is collected from the same prescreening dataset sourced from the fragmentation pipeline illustrated in FIG. 2. The Worst Pool (WP) runs use the worst 1000 fragments by ligand binding affinity. The Large Pool (LP) runs use all fragments, regardless of binding affinity.

The Unprioritized (U) and Prioritized (P) runs use the same dataset of fragments, where up to 1000 of the best fragments per unique amino acid are included in the pool. The distinction between the two runs is that the P runs pair fragments with interacting amino acids sourced from PLIP. This difference tests the effect of matching fragments with associated amino acids rather than randomly assigning fragments. The WP, LP, and U runs all add random fragments within the pool, while the P runs target fragments towards specific amino acids.

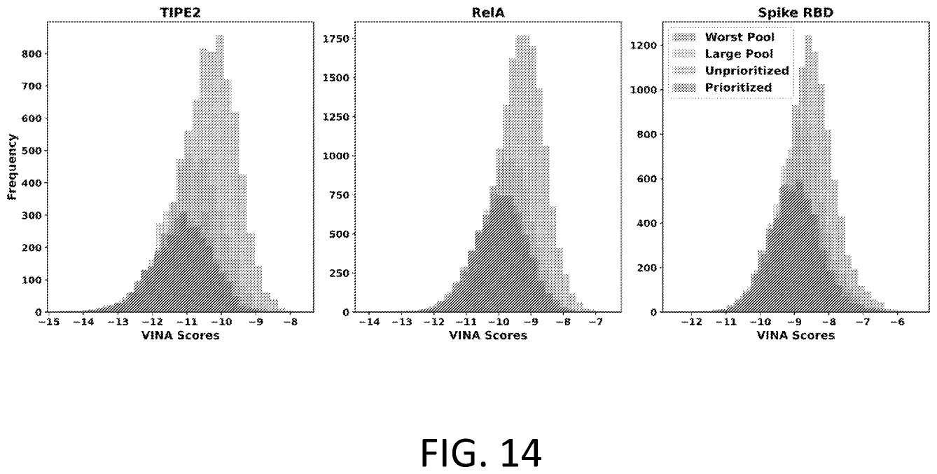

The histograms in FIG. 14 highlight the final run results for each protein target. The max ligand size producible by the algorithm is 700 g/mol. Percentile scores and top median pools are highlighted because they tend to be where leads are chosen from. The percentile scores describe how good the distribution of ligands is towards the top of the results, while the top median scores describe how improved the very best ligands are.

The P and U plots are shifted left relative to LP and WP runs, indicating a bias towards producing ligands with better binding affinities. For all protein targets, the median and mean VINA scores improve in order of WP, LP, U, and P. In addition, the 95th, 97th, and 99th percentile scores highlight a significant decrease in VINA scores at the top end of each dataset, with significant improvements in VINA scores in the P and U runs relative to the LP and WP runs. However, the P and U runs tend to have percentile scores within ±0.01 kcal/mol of each other, indicating negligible differences in score between one another. The Top 50 to 10 Median scores for RelA and Spike RBD show a similar pattern, where median scores improve from WP to LP to U/P. Interestingly, the ligands generated for the TIPE2 target did not show a similar trend in score, with LP, U, and P Top 50 to 10 median scores demonstrating no clear trend between runs. The Top 10 Median LP run even outperformed the U and P runs at −14.21 kcal/mol compared to −14.04 kcal/mol and −13.98 kcal/mol respectively.

Outlier ligands are often produced by chance. Addition of a fragment to a given carbon may on rare occasions significantly improve binding affinity over targeted efforts to improve the overall distribution of ligands. Therefore, ligands towards the top of the results do not follow the trend of improving binding affinities in the order of WP, LP, and U/P.

Although the Large Pool and Worst Pool runs have lower median binding affinities, they overall produce significantly more ligands relative to the Prioritized and Unprioritized runs, as per the Counts column of each run in Table 7 for each of the protein targets. This is expected as fragments towards the top of the fragment dataset tend to have larger molecular weights. Each addition in the U and P runs increases the molecular higher than an addition in the WP and LP runs, which causes the U and P runs to reach the max molecular weight of 700 g/mol more rapidly. This causes the U and P runs to produce fewer unique ligands than the LP and WP runs.

TABLE 7
Iterative Run Statistics

| | | | | |
|---|---|---|---|---|
| Run | Worst Pool | Large Pool | Unprioritized | Prioritized |

(a) TIPE2 (in kcal/mol)

| | | | | |
|---|---|---|---|---|
| Median | −10.34 | −10.86 | −11.04 | −11.12 |
| Mean | −10.41 | −10.91 | −11.09 | −11.15 |
| Mode | −10.19 | −10.59 | −10.53 | −11.3 |
| Count | 7480 | 4605 | 3318 | 3188 |
| 95th Percentile | −11.89 | −12.37 | −12.58 | −12.59 |
| 97th Percentile | −12.18 | −12.61 | −12.85 | −12.84 |
| 99th Percentile | −12.78 | −13.22 | −13.38 | −13.33 |
| Top 50 Median | −13.3 | −13.43 | −13.48 | −13.46 |
| Top 20 Median | −13.58 | −13.82 | −13.84 | −13.78 |
| Top 10 Median | −13.86 | −14.2 | −14.04 | −13.98 |
| Best Ligand | −14.45 | −14.69 | −14.46 | −14.37 |

(b) RelA (in kcal/mol)

| | | | | |
|---|---|---|---|---|
| Median | −9.28 | −9.75 | −9.92 | −9.94 |
| Mean | −9.31 | −9.79 | −9.96 | −9.97 |
| Mode | −10.05 | −10.11 | −10.01 | −10.19 |
| Count | 15328 | 9218 | 6710 | 6356 |
| 95th Percentile | −10.53 | −11.13 | −11.34 | −11.33 |
| 97th Percentile | −10.72 | −11.38 | −11.54 | −11.55 |
| 99th Percentile | −11.05 | −11.83 | −11.94 | −12.02 |
| Top 50 Median | −11.67 | −12.26 | −12.38 | −12.45 |
| Top 20 Median | −11.98 | −12.5 | −12.78 | −12.84 |
| Top 10 Median | −12.28 | −12.72 | −13.0 | −13.2 |
| Best Ligand | −12.74 | −13.27 | −13.74 | −14.06 |

(c) Spike RBD (in kcal/mol)

| | | | | |
|---|---|---|---|---|
| Median | −8.54 | −8.84 | −8.95 | −9.01 |
| Mean | −8.53 | −8.83 | −8.95 | −9.0 |
| Mode | −8.525 | −10.21 | −10.08 | −10.02 |
| Count | 10148 | 6629 | 5362 | 4845 |
| 95th Percentile | −9.66 | −10.03 | −10.19 | −10.23 |
| 97th Percentile | −9.84 | −10.21 | −10.41 | −10.41 |
| 99th Percentile | −10.17 | −10.52 | −10.74 | −10.78 |
| Top 50 Median | −10.59 | −10.77 | −10.98 | −10.98 |
| Top 20 Median | −10.81 | −10.98 | −11.3 | −11.24 |
| Top 10 Median | −11.0 | −11.14 | −11.48 | −11.46 |
| Best Ligand | −11.17 | −11.97 | −11.99 | −12.49 |

Comparison of Prescreening and Optimization Methodology with Other Genetic and Deep Learning Methods

In addition to the above experiments, trials of other ligand optimization packages are shown here to compare the effectiveness of the described state-of-art genetic and iterative (machine learning) methodologies. One exemplary package that takes a genetic approach is AutoGrow4. Due to limitations in ligand pool size, for the trials shown here, AutoGrow4 is fed a random sample of 1000 source ligands from the top 10000 ligands used by the fragmentation pipeline and was ran 10 times. All default variables and packages were used, and the file conversion package selected is obabel.

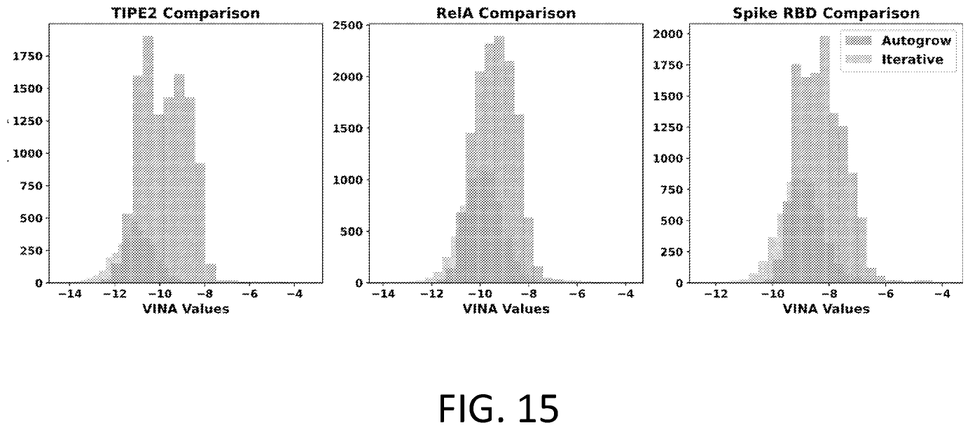

Each run is allowed to run for 30 generations, at which scores between generations tend to plateau. The same receptors and search boxes used by the genetic and iterative code are supplied to AutoGrow4. The histograms in FIG. 15 highlight significantly higher binding affinities for ligands generated by AutoGrow4 relative to binding affinities generated by the iterative methodology. This is further highlighted in the tables, which show significant improvements in binding affinity in the percentile score columns and the top ligand median score columns.

Interestingly, AutoGrow4 appeared to generate a better overall median score in the RelA trial. The best ligands produced by AutoGrow4 in all 10 runs for each target protein had VINA scores of −12.6, −11.8, and −10.3 kcal/mol for TIPE2, RelA, and Spike RBD respectively, while the best iterative ligands from the Prioritized runs were −14.37, −14.06, and −12.49 kcal/mol. The second-stage iterative optimization of the proposed approach appears to produce significantly fewer ligands than AutoGrow4, as indicated by the counts in Table 8. This is attributable to the iterative approach only being able to add fragments onto ligands, which means it will hit the molecular weight ceiling faster than an exclusively genetic approach like AutoGrow4. AutoGrow4 can mix and match substructures within a given population of molecules, allowing for more combinations and hence more unique ligands.

TABLE 8
AutoGrow4 Comparison (in kcal/mol)

| | TIPE2 | | RelA | | Spike RBD | |
|---|---|---|---|---|---|---|
| Run | AutoGrow4 | Prioritized | AutoGrow4 | Prioritized | AutoGrow4 | Prioritized |
| Median | −9.8 | −11.12 | −9.4 | −9.94 | −8.3 | −9.01 |
| Mean | −9.77 | −11.15 | −9.41 | −9.97 | −8.25 | −9.0 |
| Mode | −10.7 | −11.3 | −9.1 | −10.19 | −8.5 | −10.02 |
| Count | 11142 | 3188 | 13760 | 6356 | 12215 | 4845 |
| 95th Percentile | −11.3 | −12.59 | −10.7 | −11.33 | −9.4 | −10.23 |
| 97th Percentile | −11.5 | −12.84 | −10.8 | −11.55 | −9.5 | −10.41 |
| 99th Percentile | −11.8 | −13.33 | −11.1 | −12.02 | −9.7 | −10.78 |
| Top 50 Median | −12.2 | −13.46 | −11.4 | −12.45 | −9.9 | −10.98 |
| Top 20 Median | −12.4 | −13.78 | −11.6 | −12.84 | −10.0 | −11.24 |
| Top 10 Median | −12.45 | −13.98 | −11.7 | −13.2 | −10.1 | −11.46 |
| Best Ligand | −12.6 | −14.37 | −11.8 | −14.06 | −10.3 | −12.49 |

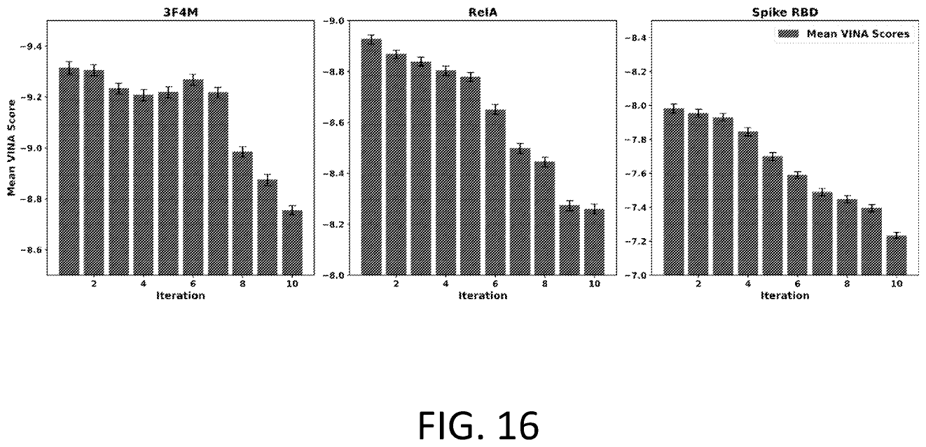

The proposed approach is also compared to DeepFrag, a deep learning approach which aims to predict the best fragment to add to a ligand within a binding pocket. The default fragments in the DeepFrag library are used in this comparison, and 10 runs are completed for each target. To create a fair comparison, each iteration, the best ligand proposed by DeepFrag is used as the input ligand for the next iteration. 10 iterations are completed per run. The intermediate ligands produced by DeepFrag are combined into one dataset which is sorted by DeepFrag scoring function. Due to computational constraints, a sample of the best ten thousand ligands from this combined dataset are ran through Autodock VINA. A histogram comparison would be ineffective at comparing the entire population of ligands generated by the proposed methodology to a sample of the DeepFrag results, so a bar plot is used to represent the results in FIG. 16.

The trend in the bar plots in FIG. 16 show that DeepFrag appears to produce poor ligands for the target binding pockets. Even when comparing best scores, DeepFrag produces significantly worse scores than the best scores of the proposed iterative methodology, at −14.37, −14.06, and −12.49 kcal/mol for TIPE2, RelA, and Spike RBD respectively. Interestingly, the average scores tend to trend down, indicating that DeepFrag may not be optimized to improve binding affinity generated by Autodock VINA. This may be caused by DeepFrag being over-fitted to the training set used, limiting extension to other protein targets like the ones presented in this study. Because the model is not tuned for the tested protein targets, the ligands generated drift into higher Autodock VINA scores, indicating decreased (poorer) binding affinities.

### Evaluating Multi-Objective Optimization for Druglikeness and Binding Affinity Based on Prescreening Information

To show that the proposed method can also account for drug likeness properties in addition to binding affinity, and still produce viable candidate ligands, a straightforward modification can be made to render the optimization multi-objective. Specifically, in addition to evaluating binding affinity using Autodock VINA, the genetic and iterative algorithms contain an optional multi-objective scoring function. The multi-objective scoring function considers QED score, a single metric generated from drug desirability functions, in addition to VINA binding scores. This scoring function can be customized depending on a user's optimization interests. The algorithm combines a ligand's VINA and QED z-scores into a single value, giving equal weight to both. The z-scores are calculated relative to the original ligand dataset from which the fragments were sourced. This methodology ensures that marginal gains in VINA performance do not significantly reduce drug likeness.

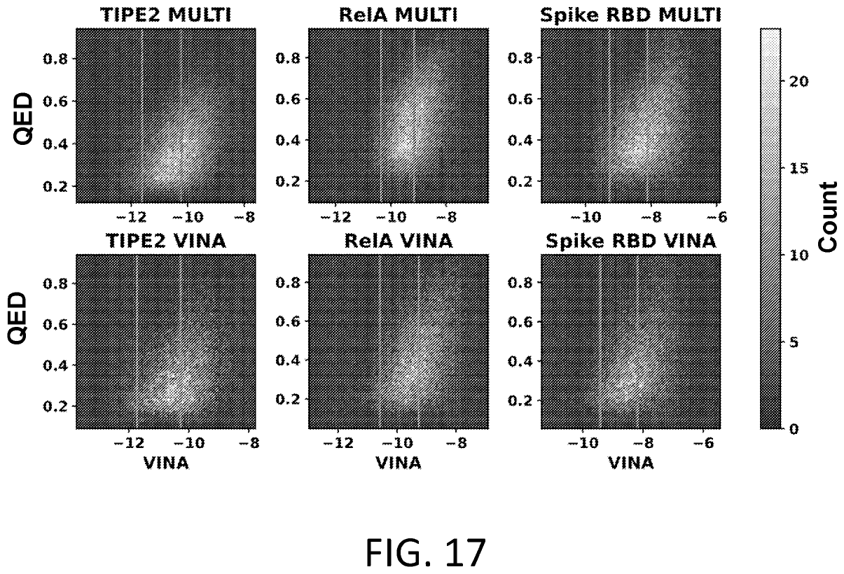

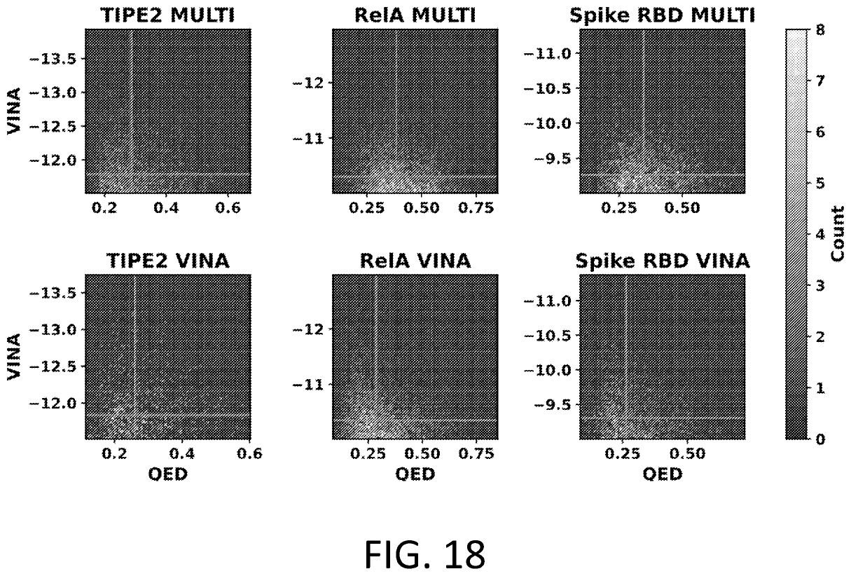

The graphs in FIG. 17 compare the generated ligands using the multi-objective approach compared to a VINA prioritization approach. Both approaches are ran on the same set of starter ligands generated from the genetic algorithm, which is ran using the multiobjective evaluation function. Table 9 summarizes the statistics for each run for the respective protein targets. The plots in FIG. 17 demonstrate that the multi-objective runs produces similar VINA score distributions to the VINA prioritization runs. Although the multi-objective prioritization produces slightly worse percentile scores, it produced better Top 50, 20, and 10 median scores during the TIPE2 runs and a better Top 10 median score during the Spike RBD run, as shown in Table 9. FIG. 18 indicates that the multi-objective function produces ligands with similar binding affinities to the VINA prioritization, while significantly improving QED scores, even at the top of the datasets where VINA scores tend to be improved but QED scores tend to be lower. Similar to the Large Pool and Worst Pool trials described above, the multi-objective runs produced far more ligands than the VINA prioritization runs, for example, as indicated by the counts in Table 9. However, both trials used the same fragment pools. The reason for this difference is that the multi-objective trials account for drug desirability, which tends to prefer smaller ligands. If a given iteration produces a marginal gain in VINA score but a large gain in molecular weight, the algorithm will backtrack as the multi-objective score will not have improved, even if the VINA score did. Therefore, more unique ligands are produced as each iteration needs to improve VINA score significantly enough to outweigh any decreases in QED score.

### Identifying Structures for Potential Candidate Ligands

To illustrate the kinds of structures that are produced as a result of the proposed pipeline and optimization methods, the structural formula and SMILES strings of three potential candidate ligands for each of the targets analyzed herein (TIPE2, RelA, and Spike RBD) are shown in Table 10 and accompanying FIG. 11. These ligands, for example, may be evaluated in future in vitro studies for binding affinity and druggability.

TABLE 9
Multi-objective Iterative Run Statistics (in kcal/mol)

| | TIPE2 | | RelA | | Spike RBD | |
|---|---|---|---|---|---|---|
| Run | MULTI | VINA | MULTI | VINA | MULTI | VINA |
| Median | −10.23 | −10.27 | −9.16 | −9.27 | −8.12 | −8.18 |
| Mean | −10.24 | −10.29 | −9.17 | −9.3 | −8.12 | −8.19 |
| Mode | −10.22 | −10.19 | −10.01 | −10.07 | −8.177 | −8.398 |
| Count | 24748 | 7784 | 24083 | 11886 | 19440 | 9041 |
| 95th Percentile | −11.63 | −11.73 | −10.36 | −10.58 | −9.28 | −9.42 |
| 97th Percentile | −11.83 | −11.94 | −10.55 | −10.77 | −9.43 | −9.62 |
| 99th Percentile | −12.25 | −12.31 | −10.9 | −11.17 | −9.71 | −9.91 |
| Top 50 Median | −13.0 | −12.73 | −11.57 | −11.73 | −10.2 | −10.28 |
| Top 20 Median | −13.27 | −12.99 | −11.87 | −12.03 | −10.39 | −10.42 |
| Top 10 Median | −13.5 | −13.16 | −12.0 | −12.29 | −10.62 | −10.56 |
| Best Ligand | −13.93 | −13.74 | −12.96 | −12.98 | −11.34 | −11.37 |

The candidate structures shown here are selected from the results of both the default-objective and multi-objective functions described in previous sections. The criteria used to select potential exemplary candidates to display in Table 10 are (i) to minimize binding affinity as predicted by Autodock VINA (specifically targeting predicted affinities of less than −13 kcal/mol or as close as possible where targets were not found in that range), (ii) estimated solubility (ESOL) scores indicating moderate solubility or better calculated to be between −4 and −6 using the method described in, and (iii) molecular weights of less than 700 g/mol. Additional quantitative values for drug-likeness properties are predicted by SwissADME and shown in Table 10.

TABLE 10
Properties Molecular Structures of Potential Candidate Ligands
Generated by the FDSL and Two-Stage Optimization Pipeline^{a}

| Compound No. and Binding | MW | | | TPSA | Log Kp | | |
|---|---|---|---|---|---|---|---|
| Affinity (kcal/mol) | (g/mol) | ESOL | MLogP | (Å²) | (cm/s) | GI | BBB |
| (i), −13.04 | 557.64 | −5.82 | 5.28 | 70.56 | −6.76 | High | Yes |
| (ii), −12.85 | 555.65 | −5.81 | 4.07 | 97.24 | −7.14 | High | No |
| (iii), −12.40 | 602.72 | −5.77 | 3.42 | 78.09 | −7.44 | High | No |
| (iv), −12.83 | 625.68 | −5.60 | 4.59 | 129.87 | −7.97 | High | No |
| (v), −12.29 | 584.67 | −4.30 | 3.41 | 124.32 | −8.87 | High | No |
| (vi), −12.18 | 548.55 | −5.41 | 2.51 | 129.03 | −7.42 | High | No |
| (vii), −11.40 | 597.62 | −4.61 | 2.69 | 178.92 | −8.75 | Low | No |
| (viii), −11.24 | 676.60 | −4.47 | 0.43 | 237.15 | −9.93 | Low | No |
| (ix), −11.12 | 552.54 | −5.65 | 4.94 | 135.21 | −7.55 | Low | No |

^{a}Structures corresponding to compounds (i) through (ix) are depicted in FIG. 11.

The Fragments from Ligands Drug Discovery (FDSL) pipeline can significantly improve computational ligand design and optimization by prioritizing fragments based on the results of initial virtual screening. By prioritizing fragments with higher potentials for success, the FDSL pipeline not only increases the efficiency of the subsequent drug design process but also it towards yielding compounds with optimal binding affinities and drug-like properties. Moreover, the FDSL pipeline includes ligand-binding domain analysis, such that key carbons for binding are identified and prioritized during the iterative step, thereby improving optimization as well.

Specifically, the results show that fine-tuned fragment pools, including fragments involving ligands with lower binding affinities, produce larger shares of ligands with good binding affinities relative to pools without prioritization. Additionally, the similar performance of the Prioritized trials (fragment pools associated by amino acid) and Unprioritized trials (Prioritized fragment pools without amino acid association) hints at a minimal influence of specifying fragments to specific amino acids during the fragment addition process. Outlier ligands with significant performance often emerge due to chance, which blurs the observable trends at the top tier of each resulting dataset.

This can be attributed to fragments within the Large Pool (pool with all fragments) and Worst Pool (pool with worst 1000 fragments by binding affinity) that significantly improve binding affinity in contexts not apparent in the source ligand dataset. Furthermore, fragments included in the Prioritized and Unprioritized pools with higher associated binding affinities tend to be heavier, hitting the maximum molecular weight of 700 g/mol in fewer iterations and thus producing fewer unique ligands than the Large Pool and Worst Pool runs. This upper limit is selected to avoid limiting max ligand sizes to 500 g/mol according to Lipinski's Rule of Five, which, as others have pointed out, are likely outdated and, if used strictly, will filter out potential solid leads. Raising the upper limit beyond 700 g/mol may result in ligands displaying stronger binding affinities.

These ligands would be better suited for the large binding pockets of the target proteins under consideration. However, it is important to note that this limit is chosen to enhance computational efficiency. Using larger ligands in Autodock VINA significantly increases the computational time required, which may limit the number of unique ligands screened within a given timeframe. The proposed method is tested on three distinct types of targets, each with its unique challenges. First, we tested protein targets associated with solid cancer tumorigenesis, aiming for better cancer treatments. Our second design target relates to the issue of antimicrobial resistance, a significant concern as rising resistance could render basic infections untreatable.

Third, we applied the proposed method to the spike protein Receptor Binding Domain (RBD) of COVID-19, where inhibiting this domain might prevent the virus from entering human cells, offering a potential therapeutic avenue. The robustness of this approach is further reinforced by its capacity to handle the varied geometries, binding conditions, and chemical conditions presented by these targets. Navigating different geometries means understanding diverse target structures, while adapting to unique binding conditions and varying chemical environments showcases the method's versatility.

The methodology yields ligands with enhanced binding affinities when compared to other methods. This improvement may be due to the capability of the genetic and iterative algorithms adapted for the FDSL pipeline described herein to accommodate larger source ligand datasets. In contrast, AutoGrow4 and DeepFrag have limitations in terms of source ligand dataset size. The scalability of the proposed methodology enables more extensive exploration and analysis of the chemical space within the binding pocket prior to ligand generation.

Scalability, in turn, makes it possible for the genetic and iterative algorithms with an even greater amount of information for constructing new ligands and fine-tuning them towards improved binding affinities than described herein. Balancing optimal binding affinity with favorable drug-likeness properties is essential for successful lead identification in drug discovery. The method as currently developed employs Quantitative Estimate of Druglikeness (QED) scores. The results demonstrate that employing a multi-objective evaluation can produce candidate leads that can have superior druglikeness properties, such as solubility, with strong binding affinity. In particular, multi-objective prioritization produces a more diverse pool of ligands compared to VINA prioritization.

The generated ligands demonstrate significant improvements in QED scores with minimal losses in binding affinity. Moreover, by prioritizing QED scores, modest improvements in binding affinity do not override significant decreases in drug-likeness, resulting in ligands with both improved binding affinities and drug-likeness. Notably, the leads in Table 10 have ESOL scores between −4 and −6, indicating moderate solubility. To prepare for in vitro studies, polar groups may be manually added to further improve solubility, though these changes may affect predicted binding affinity. Other computational estimations of solubility, such as M Log P, can also be assessed to determine if log P scores are less than 5, which agrees with Lipinski's rule of fives.

Although the Prioritized runs did not yield significant improvements in binding affinity relative to the Unprioritized runs, further pool screening should be completed in future studies to determine if amino acid matching can yield stronger ligands. For instance, matching fragment properties to amino acid properties instead of exclusively relying on PLIP analysis may yield more optimal fragment amino acid pairings, improving ligand binding affinity upon addition. Future studies may also consider adding other objectives for optimization.

Finally, the proposed method relies on computationally predicted rather than actual experimental data on the initial ligand population—while still providing useful information to guide the drug design and optimization process. Not only is the prescreening data generated in silico, thereby avoiding costs and complexity of in vitro screening, but the prescreening is also done using relatively low computational-cost and scalable computer docking methods, as opposed to more costly and less scalable molecular dynamics methods. Accordingly, the FDSL and optimization methods described herein are highly scalable. To further scalability, the code developed to implement both the genetic and iterative optimization stages herein is fully parallelized and can be readily executed across multiple processors in a computing environment as the ligand population and fragment pool increases.

### Computational Ligand Design

A basic prioritized search for new ligands based on the fragment library generated by the FDSL-DD is described as follows and in FIG. 3. As an initial step, fragments are sorted based on the inferred binding affinity of their parent ligand, as shown in FIG. 2. Additional sorting can be performed—for example, fragments binding to particular regions (as shown in FIG. 2) may be placed in different pools, and fragments may be drawn from them according to the scheme shown in FIG. 3 and described here. A set of new ligands is generated based on a triangular number sequence. As shown in FIG. 3, all possible ways of summing the numbers m through n, where m is the first ranked fragment (generally m=1) and n is the last ranked considered for combination within the ranked list of fragments are generated. For example, for m=1 and n=5, the combinations are 1+2, 1+3, 1+4, and 2+3. That means that candidate ligands are the combination of fragments ranked 1 and 2, 1 and 3, 1 and 4, and 2 and 3 respectively. Because most fragments are between 100-300 g/mol, combinations are only added with up to 5 fragments to comply with Lipinski's 500 g/mol rule. This significantly decreases computational burden, as only a subset of ligands that can be generated with 5 operands are evaluated.

BRICS fragments are combined using BRICS rules using the BRICS. Build command in RDKit. This is possible because the MolFile or SMARTS representations of BRICS fragments will store information about the broken bonds in BRICS fragmentation in isotopes. The BRICS.Build package in RDKit can utilize the isotope information to attempt to recombine fragments in new combinations according to the information in the isotopes. If the resulting molecule can be parsed by RDKit, then it is successful potential molecule. If isotopes remain, indicating potential binding sites, then other fragments can be added on to the growing ligand. If a different computational fragmentation procedure is used, then different computational methods can be used to assemble fragments. For example, while not implemented for the results described herein, the pipeline allows for RECAP fragments, if generated, to be combined through text processing of SMILES strings by replacing the (*) wildcard character used to indicate broken bonds (i.e., open binding sites).

It is important to note that not every fragment fed into the BRICS recomposition algorithm is guaranteed to be included in the final molecule. FIG. 10 shows, for example, RAC4 and its parent fragments. As shown in FIG. 10, in the case of RAC4, the third fragment fed did not end up in the final ligand. Additionally, BRICS may repeat fragments to fill the valences of each incorporated fragment. In the case of RAC4, fragment 1 and 3 had more than one fragment end. Regardless of which fragment was used, at least one repeat of fragment 2 is necessary to fill in all valences since fragment 2 is the only fragment with only one exposed end. Although repeat fragments are not ideal from a diversity perspective, in some cases, they enable molecules with better binding affinities to be generated, like in the case of RAC4.

The resulting ligands are then evaluated based on their binding affinity using Autodock VINA. Optionally, in silico absorption, distribution, metabolism, and excretion (ADME) properties are also calculated using SwissADME, as well as other drug likeness properties such as the Lipinski's rule of 5 parameters, using built-in RDKit features in the rdkit. Chem.Lipinski module. The highest-ranking ligands based on binding affinities and ADME properties are also visualized in protein-ligand complexes with PyMOL and ChimeraX-1.2.1.

The source code for the methods described herein will be made available on request for research and educational purposes only and under a license that prohibits any commercial or third-party use.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following enumerated embodiments are provided, the numbering of which is not to be construed as designating levels of importance:

Embodiment 1 provides a computer-implemented method of drug design, the method comprising the steps of:
(a) accessing a computer model of an atomic structure of a target protein and a plurality of ligands independently docked and positioned in a binding region in the target protein, wherein each ligand in the plurality of ligands has an associated binding affinity with the target protein;
(b) fragmenting each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally joining two or more ligand fragments to form a drug, wherein a chemical structure of the drug is chemically stable and the two or more ligand fragments have fragment binding scores in the top 10% of a rank-ordered sorting of ligand binding scores of the plurality of ligand fragments.

Embodiment 2 provides the method of embodiment 1, wherein average molecular weight of the plurality of ligands is at least 350 g/mol.

Embodiment 3 provides the method of any one of embodiments 1-2, wherein the position of the plurality of ligands relative to the target protein is stored in a computer accessible database.

Embodiment 4 provides the method of any one of embodiments 1-3, further comprising associating the plurality of ligand fragments with the ligand from which they originate.

Embodiment 5 provides the method of any one of embodiments 1-4, wherein the position of the plurality of ligands fragments relative to the target protein is stored in a computer accessible database.

Embodiment 6 provides the method of any one of embodiments 1-5, wherein the ligand fragment binding score comprises one or more of mean binding affinity of the ligand fragment, median binding affinity of the ligand fragment, mode binding affinity of the ligand fragment, binding affinity of the ligand, frequency with which the fragment occurs in the plurality of ligand fragments, calculated ADME (absorption, distribution, metabolism, and excretion) properties of the ligand fragment, and deviation between the mean ligand binding affinity corresponding to a ligand fragment-subregion combination and an overall mean binding affinity of the plurality of ligands.

Embodiment 7 provides the method of any one of embodiments 1-6, wherein ligand fragments that appear more often than other ligand fragments are top lead fragments (TLF), each having a rank.

Embodiment 8 provides the method of any one of embodiments 1-7, wherein the computational joining of two or more ligand fragments comprises linking two or more TLFs for continuous subregions of the target protein.

Embodiment 9 provides the method of any one of embodiments 1-8, wherein the computational joining comprises linking two or more TLFs such that a sum of the ranks of the TLFs is an integer from 3 to 10.

Embodiment 10 provides the method of any one of embodiments 1-9, wherein the fragments are selected at least in part based on a predicted subpocket location of the fragment.

Embodiment 11 provides the method of any one of embodiments 1-10, wherein the fragments are selected at least in part based on amino acids in the target protein that are computationally predicted to bind to the ligand fragment.

Embodiment 12 provides a system configured to implement a computer-implemented method of drug design, the system comprising:
an optional display screen; and
a computer system comprising one or more processors;
the computer system being configured and adapted to implement deep reinforcement learning,
the computer system being further configured and adapted to be provided with objectives for a therapeutically effective drug,
wherein the one or more processors are configured to execute a set of instructions that:
(a) provide a computer model of an atomic structure of a target protein and a plurality of ligands independently docked in a binding region in the target protein, wherein each ligand in the plurality of ligands has an associated binding affinity with the target protein;
(b) fragment each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally join two or more ligand fragments to form a drug, wherein a chemical structure of the drug is chemically stable and the two or more ligand fragments have fragment binding scores in the top 10% of a rank-ordered sorting of ligand binding scores of the plurality of ligand fragments.

Embodiment 13 provides the system of embodiment 12, wherein the computer system is further configured and adapted to achieve a high binding affinity with the protein target.

Embodiment 14 provides the system of any one of embodiments 12-13, wherein the computer system is further configured and adapted to achieve drugs which are highly soluble and minimally hydrophobic.

Embodiment 15 provides the system of any one of embodiments 12-14, wherein the computer system is further configured and adapted to learn how to optimize drug design by automatically reinforcing designs that better meet objectives in a deep reinforcement learning training process.

Embodiment 16 provides a computer-readable recording medium storing instructions to execute the method of any one of embodiments 1-11.

Embodiment 17 provides a computer-implemented method of drug design, the method comprising:
(a) accessing a computer model of a target protein and computer models of a plurality of ligand structures;
(b) obtaining a predicted binding affinity between each of the ligands and the target protein;
(c) providing protein-ligand bond profiling data based on computational predictions on how each of the plurality of ligand structures binds with the target protein;
(d) fragmenting the plurality of ligand structures into computer models of ligand fragments; and
(e) providing scores for each of the ligand fragments using the predicted binding affinity and the protein-ligand bond profiling data.

Embodiment 18 provides the method of embodiment 17, further comprising:
(f) providing a fragment database based on the scores for each of the ligand fragments.

Embodiment 19 provides the method of any one of embodiments 17-18, further comprising:
(g) utilizing the fragment database to design drug candidates in silico.

Embodiment 20 provides the method of any one of embodiments 17-19, further comprising:
(h) providing a computer system for implementing steps of (a)-(g), the computer system being configured and adapted to implement a deep reinforcement learning (Deep RL) training process.

Embodiment 21 provides the method of any one of embodiments 17-20, further comprising:
(i) providing objectives to the computer system, the objectives being defined for an effective and useful drug.

Embodiment 22 provides the method of any one of embodiments 17-21, wherein the objectives are selected from the group consisting of: high binding affinity with the protein target, high aqueous solubility, and low lipophilicity.

Embodiment 23 provides the method of any one of embodiments 17-22, wherein the computer system is configured and adapted to learn the best assemblies of fragments to formulate synthetic ligands that meet the provided objectives.

Embodiment 24 provides the method of any one of embodiments 17-23, wherein the computer system is configured and adapted to learn how to optimize drug design by automatically reinforcing designs that correspond more closely to the provided objectives in the Deep RL training process.

Embodiment 25 provides computer-implemented method of drug design, the method comprising:
(a) accessing a computer model of an atomic structure of a target protein and a plurality of ligands independently docked in a binding region in the target protein, wherein each ligand in the plurality of ligands has an associated binding affinity with the target protein;
(b) fragmenting each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally joining two or more ligand fragments to form a drug, wherein a chemical structure of the drug is chemically stable and the two or more ligand fragments have fragment binding scores above a certain threshold of a rank-ordered sorting of ligand binding scores of the plurality of ligand fragments.

Embodiment 26 provides the method of embodiment 25, wherein the certain threshold is set at the 70th percentile.

Embodiment 27 provides a computer-implemented method of drug design, the method comprising:
(a) accessing a computer model of an atomic structure of a target protein and a plurality of ligands independently docked in a binding region in the target protein, wherein each ligand in the plurality of ligands has an associated binding affinity with the target protein;
(b) fragmenting each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally joining two or more ligand fragments to form a drug, wherein a chemical structure of the drug is chemically stable and the two or more ligand fragments have fragment binding scores in the top of a certain percentage of a rank-ordered sorting of ligand binding scores of the plurality of ligand fragments, wherein the certain percentage is dependent on a number of computationally combined compounds.

1. A computer-implemented method of drug design, the method comprising the steps of:
(a) accessing a computer model of an atomic structure of a target protein and a plurality of ligands independently docked and positioned in a binding region in the target protein, wherein each ligand in the plurality of ligands has an associated binding affinity with the target protein;
(b) fragmenting each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally joining two or more ligand fragments to form a drug, wherein a chemical structure of the drug is chemically stable and the two or more ligand fragments have fragment binding scores in the top 10% of a rank-ordered sorting of ligand binding scores of the plurality of ligand fragments.

2. The method of claim 1, wherein average molecular weight of the plurality of ligands is at least 350 g/mol.

3. The method of claim 1, wherein the position of the plurality of ligands relative to the target protein is stored in a computer accessible database.

4. The method of claim 1, further comprising associating the plurality of ligand fragments with the ligand from which they originate.

5. The method of claim 1, wherein the position of the plurality of ligands fragments relative to the target protein is stored in a computer accessible database.

6. The method of claim 1, wherein the ligand fragment binding score comprises one or more of mean binding affinity of the ligand fragment, median binding affinity of the ligand fragment, mode binding affinity of the ligand fragment, binding affinity of the ligand, frequency with which the fragment occurs in the plurality of ligand fragments, calculated ADME (absorption, distribution, metabolism, and excretion) properties of the ligand fragment, and deviation between the mean ligand binding affinity corresponding to a ligand fragment-subregion combination and an overall mean binding affinity of the plurality of ligands.

7. The method of claim 6, wherein ligand fragments that appear more often than other ligand fragments are top lead fragments (TLF), each having a rank.

8. The method of claim 7, wherein the computational joining of two or more ligand fragments comprises linking two or more TLFs for continuous subregions of the target protein.

9. The method of claim 7, wherein the computational joining comprises linking two or more TLFs such that a sum of the ranks of the TLFs is an integer from 3 to 10.

10. The method of claim 1, wherein the fragments are selected at least in part based on a predicted subpocket location of the fragment.

11. The method of claim 1, wherein the fragments are selected at least in part based on amino acids in the target protein that are computationally predicted to bind to the ligand fragment.

12. A system configured to implement a computer-implemented method of drug design, the system comprising:
a computer system comprising one or more processors;
the computer system being configured and adapted to implement deep reinforcement learning,
the computer system being further configured and adapted to be provided with objectives for a therapeutically effective drug,
wherein the one or more processors are configured to execute a set of instructions that:
(a) provide a computer model of an atomic structure of a target protein and a plurality of ligands independently docked in a binding region in the target protein, wherein each ligand in the plurality of ligands has an associated binding affinity with the target protein;
(b) fragment each of the plurality of ligands into a plurality of ligand fragments, wherein each ligand fragment in the plurality of ligand fragments has an associated fragment binding score, wherein the fragment binding score corresponds to an interaction energy between the ligand fragment and at least one sub-region of the target protein with which the ligand fragment interacts; and
(c) computationally join two or more ligand fragments to form a drug, wherein a chemical structure of the drug is chemically stable and the two or more ligand fragments have fragment binding scores in the top 10% of a rank-ordered sorting of ligand binding scores of the plurality of ligand fragments.

13. The system of claim 12, wherein the computer system is further configured and adapted to achieve a high binding affinity with the protein target.

14. The system of claim 12, wherein the computer system is further configured and adapted to achieve drugs which are highly soluble and minimally hydrophobic.

15. The system of claim 12, wherein the computer system is further configured and adapted to learn how to optimize drug design by automatically reinforcing designs that better meet objectives in a deep reinforcement learning training process.

16. A computer-readable recording medium storing instructions to execute the method of claim 1.

17. A computer-implemented method of drug design, the method comprising:
(a) accessing a computer model of a target protein and computer models of a plurality of ligand structures;
(b) obtaining a predicted binding affinity between each of the ligands and the target protein;
(c) providing protein-ligand bond profiling data based on computational predictions on how each of the plurality of ligand structures binds with the target protein;
(d) fragmenting the plurality of ligand structures into computer models of ligand fragments; and
(e) providing scores for each of the ligand fragments using the predicted binding affinity and the protein-ligand bond profiling data.

18. The method of claim 17, further comprising:
(f) providing a fragment database based on the scores for each of the ligand fragments.

19. The method of claim 18, further comprising:
(g) utilizing the fragment database to design drug candidates in silico.

20. The method of claim 19, further comprising:
(h) providing a computer system for implementing steps of (a)-(g), the computer system being configured and adapted to implement a deep reinforcement learning (Deep RL) training process.

21. The method of claim 20, further comprising:
(i) providing objectives to the computer system, the objectives being defined for an effective and useful drug.

22. The method of claim 21, wherein the objectives are selected from the group consisting of: high binding affinity with the protein target, high aqueous solubility, and low lipophilicity.

23. The method of claim 21, wherein the computer system is configured and adapted to learn how to optimize drug design by automatically reinforcing designs that correspond more closely to the provided objectives in the Deep RL training process.
